(19) United States
(12) Patent Application Publication
(10) Pub. No.: US 2025/0152749 A1
(43) Pub. Date: May 15, 2025

(54) ASSESSMENT OF ADVERSE EVENTS INDUCED BY ADMINISTRATION OF ANTITUMOR DRUG AND/OR IMMUNE CHECKPOINT INHIBITOR BY USING DISEASE MODEL MOUSE

(71) Applicant: TOHOKU UNIVERSITY, Miyagi (JP)

(72) Inventors: Tetsuya KODAMA, Miyagi (JP); Shiro MORI, Miyagi (JP)

(73) Assignee: TOHOKU UNIVERSITY, Miyagi (JP)

(21) Appl. No.: 18/839,492

(22) PCT Filed: Feb. 15, 2023
(86) PCT No.: PCT/JP2023/005345

§ 371 Date: Aug. 19, 2024

(30) Foreign Application Priority Data
Feb. 22, 2022 (JP) 2022-026024

(51) Int. Cl. A61K 49/00; A01K 67/0275; G01N 33/50

(52) U.S. Cl. CPC A61K 49/0008; A01K 67/0275; G01N 33/5088; G01N 33/5091; A01K 2227/105; A01K 2267/03

(57) ABSTRACT

Provided is a method of assessing an adverse effect induced by an antitumor dug and/or an immune checkpoint inhibitor, the method including: administering the antitumor drug and/or the immune checkpoint inhibitor to a model mouse that is any one of (i) one or two or more kinds selected from the group consisting of an MXH7/Mon/Ipr mouse, an MXH10Mo/Ipr mouse, an MXH28/Mo/Ipr mouse, an MXH36/Mo/Ipr mouse, an MXH41/Mo/Ipr mouse, an MXH43/Mo/Ipr mouse, an MXH51/Mon/Ipr mouse, and an MXH54/Mo/Ipr mouse, or (ii) one or two or more kinds selected from the group consisting of: an McH/Mo/Ank.Y^{C3H} mouse, an McH/Mo/Ank.Y^{MRL} mouse, and an McH/Mo/Ipr/RA1 mouse, and assessing the adverse effect induced by the antitumor drug and/or the immune checkpoint inhibitor.

### TECHNICAL FIELD

The present invention relates to an assessment of an adverse effect induced by administration of an antitumor drug and/or an immune checkpoint inhibitor by using a disease model mouse, and more specifically, to an assessment of an adverse effect induced by administration of an antitumor drug and/or an immune checkpoint inhibitor by using a collagen disease model mouse.

### BACKGROUND ART

It has been well known that an anticancer drug often induces not only a pharmacological action but also a side effect, and that the use of an immune checkpoint inhibitor serving as one kind of anticancer drug develops an immune-related adverse effect (irAE). For example, it has been recognized that the administration of an anti-PD antibody or an anti-CTLA-4 antibody serving as an immune checkpoint inhibitor, or a combination thereof to a cancer patient causes an immune-related adverse effect in the patient's skin, endocrine system, stomach, liver, lung, or kidney at a certain ratio (Non-patent Literature 1).

A method by which an adverse effect accompanying the administration of an antitumor drug and/or an immune checkpoint inhibitor can be simply assessed has been required.

### CITATION LIST

### Non-Patent Literature

NPL 1: Kang et al. Trends in Immunology. 2021; 42:292-311

### SUMMARY OF INVENTION

### Technical Problem

An object of the present invention is to provide a method of assessing an adverse effect induced by an antitumor drug and/or an immune checkpoint inhibitor, a method of identifying a chromosome region related to an adverse effect induced by the administration of an antitumor drug and/or an immune checkpoint inhibitor, and a method of assessing an effect of an antitumor drug and/or an immune checkpoint inhibitor in each of which a collagen disease model mouse is used.

### Solution to Problem

The present invention includes embodiments described below.
Item 1. A method of assessing an adverse effect induced by an antitumor drug and/or an immune checkpoint inhibitor, the method including:
administering the antitumor drug and/or the immune checkpoint inhibitor to a model mouse that is any one of
(i) one or two or more kinds selected from the group consisting of: an MXH7/Mo/Ipr mouse, an MXH10/Mo/Ipr mouse, an MXH28/Mo/Ipr mouse, an MXH36/Mo/Ipr mouse, an MXH41/Mo/Ipr mouse, an MXH43/Mo/Ipr mouse, an MXH51/Mo/Ipr mouse, and an MXH54/Mo/Ipr mouse, or
(ii) one or two or more kinds selected from the group consisting of: an McH/Mo/Ank.Y^{C3H} mouse, an McH/Mo/Ank.Y^{MRL} mouse, and an McH/Mo/Ipr/RA1 mouse; and assessing the adverse effect induced by the antitumor drug and/or the immune checkpoint inhibitor.
Item 2. A method of screening an antitumor drug and/or an immune checkpoint inhibitor, the method including:
administering a test substance that is the antitumor drug and/or the immune checkpoint inhibitor to a model mouse that is any one of
(i) one or two or more kinds selected from the group consisting of: an MXH7/Mo/Ipr mouse, an MXH10/Mo/Ipr mouse, an MXH28/Mo/Ipr mouse, an MXH36/Mo/Ipr mouse, an MXH41/Mo/Ipr mouse, an MXH43/Mo/Ipr mouse, an MXH51/Mo/Ipr mouse, and an MXH54/Mo/Ipr mouse, or
(ii) one or two or more kinds selected from the group consisting of: an McH/Mo/Ank.Y^{C3H} mouse, an McH/Mo/Ank.Y^{MRL} mouse, and an McH/Mo/Ipr/RA1 mouse; and assessing the presence or absence of an adverse effect in the model mouse after the administration of the test substance.
Item 3. The method according to Item 1 or 2, wherein the antitumor drug and/or the immune checkpoint inhibitor is the immune checkpoint inhibitor.
Item 4. A method of identifying a chromosome region related to an adverse effect induced by administration of an antitumor drug and/or an immunotherapy drug, the method including:
administering the antitumor drug and/or the immunotherapy drug to each of model mice each of which is any one of
(i) one or two or more kinds selected from the group consisting of: an MXH7/Mo/Ipr mouse, an MXH10/Mo/Ipr mouse, an MXH28/Mo/Ipr mouse, an MXH36/Mo/Ipr mouse, an MXH41/Mo/Ipr mouse, an MXH43/Mo/Ipr mouse, an MXH51/Mo/Ipr mouse, and an MXH54/Mo/Ipr mouse, or
(ii) one or two or more kinds selected from the group consisting of an McH/Mo/Ank.Y^{C3H} mouse, an McH/Mo/Ank.Y^{MRL} mouse, and an McH/Mo/Ipr/RA1 mouse; and
identifying a chromosome region related to a difference in adverse effect induced by the administration of the antitumor drug and/or the immunotherapy drug between the plurality of kinds of model mice.
Item 5. A method of identifying a chromosome region related to an immune adverse effect induced by administration of an immune checkpoint inhibitor, the method including:
administering the immune checkpoint inhibitor to a model mouse that is any one of
(i) one or two or more kinds selected from the group consisting of: an MXH7/Mo/Ipr mouse, an MXH10/Mo/Ipr mouse, an MXH28/Mo/Ipr mouse, an MXH36/Mo/Ipr mouse, an MXH41/Mo/Ipr mouse, an MXH43/Mo/Ipr mouse, an MXH51/Mo/Ipr mouse, and an MXH54/Mo/Ipr mouse, or
(ii) one or two or more kinds selected from the group consisting of: an McH/Mo/Ank.Y^{C3H} mouse, an McH/Mo/Ank.Y^{MRL} mouse, and an McH/Mo/Ipr/RA1 mouse; and
identifying the chromosome region related to the immune adverse effect induced by the administration of the immune checkpoint inhibitor.
Item 6. The method according to any one of Items 1 to 5, wherein the adverse effect is interstitial pneumonia, sialoadenitis, vasculitis, arthritis, glomerulonephritis, dacryoadenitis, or a combination of two or more thereof.
Item 7. A method of producing an interstitial pneumonia model mouse, the method including:
administering the immune checkpoint inhibitor to a model mouse that is any one of
(i) one or two or more kinds selected from the group consisting of: an MXH7/Mo/Ipr mouse, an MXH10/Mo/Ipr mouse, an MXH28/Mo/Ipr mouse, an MXH36/Mo/Ipr mouse, an MXH41/Mo/Ipr mouse, an MXH43/Mo/Ipr mouse, an MXH51/Mo/Ipr mouse, and an MXH54/Mo/Ipr mouse, or
(ii) one or two or more kinds selected from the group consisting of: an McH/Mo/Ank.Y^{C3H} mouse; an McH/Mo/Ank.Y^{MRL} mouse; and an McH/Mo/Ipr/RA1 mouse.
Item 8. The method according to any one of Items 1 to 7, further including inoculating a tumor to the model mouse before the administration step.
Item 9. The method according to any one of Items 1 to 8, wherein the model mouse is one or two or more kinds selected from the group consisting of: the MXH7/Mo/Ipr mouse, the MXH10/Mo/Ipr mouse, the MXH28/Mo/Ipr mouse, the MXH36/Mo/Ipr mouse, the MXH41/Mo/Ipr mouse, the MXH43/Mo/Ipr mouse, the MXH51/Mo/Ipr mouse, and the MXH54/Mo/Ipr mouse.
Item 10. The method according to any one of Items 1 to 8, wherein the model mouse is one or two or more kinds selected from the group consisting of: the McH/Mo/Ank.Y^{C3H} mouse, the McH/Mo/Ank.Y^{MRL} mouse, and the McH/Mo/Ipr/RA1 mouse.
Item 11. A model mouse suffering from interstitial pneumonia, the model mouse being any one of
(i) one or two or more kinds selected from the group consisting of: an MXH7/Mo/Ipr mouse, an MXH10/Mo/Ipr mouse, an MXH28/Mo/Ipr mouse, an MXH36/Mo/Ipr mouse, an MXH41/Mo/Ipr mouse, an MXH43/Mo/Ipr mouse, an MXH51/Mo/Ipr mouse, and an MXH54/Mo/Ipr mouse, or
(ii) one or two or more kinds selected from the group consisting of: an McH/Mo/Ank.Y^{C3H} mouse; an McH/Mo/Ank.Y^{MRL} mouse; and an McH/Mo/Ipr/RA1 mouse.

### Advantageous Effects of Invention

According to the present invention, an adverse effect or a disease state induced by an antitumor drug and/or an immune checkpoint inhibitor can be simply assessed.

### BRIEF DESCRIPTION OF DRAWINGS

FIG. 1 is an explanatory view of an MMXH/Mo/Ipr or McH/Mo mouse. Typical superficial lymph nodes include a subiliac lymph node (SiLN), a proper axillary lymph node (PLAN), and an accessory axillary lymph node (AALN).

FIGS. 2(A)-(D) show the tissue staining of an MXH51/Mo/Ipr mouse having administered thereto an anti-PD-1 antibody. FIG. 2(A) shows a normal blood vessel (EM staining),

FIG. 2(B) shows a normal kidney (HE staining), FIG. 2(C) shows a lung suffering from interstitial pneumonia (EM staining), and FIG. 2(D) shows the lung suffering from interstitial pneumonia (HE staining) (at a magnification of 20).

FIG. 3(A) is a graph for showing changes in normalized luciferase activity in a control mouse, a mouse having administered thereto an anti-CTLA-4 antibody, and a mouse having administered thereto the anti-PD-1 antibody after tumor transplantation, and FIG. 3(B) is a graph for showing changes in volume of a subiliac lymph node in the control mouse, the mouse having administered thereto the anti-CTLA-4 antibody, and the mouse having administered thereto the anti-PD-1 antibody after the tumor transplantation.

FIGS. 4(A)-(D) show the occurrence of adverse effects in the respective tissues of MXH10/Mo/Ipr model mice having administered thereto various checkpoint inhibitors. FIG. 4(A) shows interstitial pneumonia in a mouse having administered thereto the anti-PD-1 antibody, FIG. 4(B) shows vasculitis in a mouse having administered thereto the anti-CTLA-4 antibody, FIG. 4(C) shows glomerulonephritis in the mouse having administered thereto the anti-CTLA-4 antibody, and FIG. 4(D) shows sialoadenitis in the mouse having administered thereto the anti-CTLA-4 antibody. The images are each taken at a magnification of 20.

FIGS. 5(A)-(D) show interstitial pneumonia induced after the administration of the anti-PD-1 antibody in McH/Mo/Ipr/RA1 mice. The anti-PD-1 antibody was administered into a subiliac lymph node, and the organ was extracted on the 21st day. FIG. 5(A) and FIG. 5(B) show the lungs of a control group, and FIG. 5(C) and FIG. 5(D) show the lungs of an anti-PD-1 antibody-administered group. FIG. 5(A) and FIG. 5(C) are each taken at a magnification of 4 (scale: 200 μm), and FIG. 5(B) and FIG. 5(D) are each taken at a magnification of 20 (scale: 50 μm).

FIGS. 6(A) and (B) show necrotizing vasculitis after the administration of the anti-PD-1 antibody to a subiliac lymph node in an MXH41/Mo-Ipr/Ipr mouse. FIG. 6(A) shows HE staining and FIG. 6(B) shows EM staining.

### DESCRIPTION OF EMBODIMENTS

Nose et al. (2007) have previously established 15 strains of the world's first collagen disease genetically modified inbred mouse MXH-Ipr/Ipr from a mouse obtained by the mating of an MRL/MpJ-Ipr/Ipr (MRL/Ipr) mouse, which is devoid of the expression of a Fas antigen gene and shows the disease states of autoimmune diseases, such as vasculitis, glomerulonephritis, arthritis, and sialoadenitis, and a C3H/HeJ-Ipr/Ipr (C3H/Ipr) mouse that does not show any such disease state over about 10 years. The inventors of the present invention have further maintained the strains for about 10 years, and have established 8 strains of an MXH recombinant inbred mouse (MXH/Mo-Ipr/Ipr) showing the disease state of a collagen disease.

Those 8 strains of inbred mice MXH/Mo-Ipr/Ipr (MXH/Mo/Ipr) were deposited under the following names to Riken BioResource Research Center (Riken BRC).
Registration number RBRC11141 MXH28/Mo-Ipr/Ipr (MXH28/Mo/Ipr) (date of deposit: Jul. 21, 2020)
Registration number RBRC11142 MXH36/Mo-Ipr/Ipr (MXH36/Mo/Ipr) (date of deposit: Jul. 21, 2020)
Registration number RBRC11143 MXH41/Mo-Ipr/Ipr (MXH41/Mo/Ipr) (date of deposit: Jul. 21, 2020)
MXH43/Mo-Ipr/Ipr (MXH43/Mo/Ipr) (date of deposit: July 21, Registration number RBRC11144 2020)
MXH51/Mo-Ipr/Ipr (MXH51/Mo/Ipr) (date of deposit: July 21, Registration number RBRC11145 2020)
Registration number RBRC11146 MXH54/Mo-Ipr/Ipr (MXH54/Mo/Ipr) (date of deposit: Jul. 21, 2020)
Registration number RBRC11152 MXH7/Mo-Ipr/Ipr (MXH7/Mo/Ipr) (date of deposit: Jul. 21, 2020)
Registration number RBRC11153 MXH10/Mo-Ipr/Ipr (MXH10/Mo/Ipr) (date of deposit: Jul. 21, 2020)

Those 8 strains of mice are similar to, but slightly different from, each other in chromosome nucleic acid sequence. The respective mice are also different from each other in frequency at which vasculitis, glomerulonephritis, arthritis, sialoadenitis, or the like develops (Nose, Allergol Int 2007; 56:79-86), and the individual clinical features of their collagen diseases are dominated by genes different from each other.

Further, the inventors of the present invention have established the following 3 strains of a congenic mouse, which spontaneously develops arthritis, ankylosis, or the like, through the mating of an MRL/Ipr mouse and a C3H/Ipr mouse.

Those 3 strains were deposited under the following names to Riken BioResource Research Center (Riken BRC).
Registration number RBRC11158 McH/Mo-Ank.Y^{C3H} (McH/Mo/Ank.Y^{C3H}) (date of deposit: Aug. 21, 2020)
Registration number RBRC11159 McH/Mo-Ank.Y^{MRL} (McH/Mo/Ank.Y^{MRL}) (date of deposit: Aug. 21, 2020)
Registration number RBRC11160 McH/Mo-Ipr/Ipr-RA1 (McH/Mo/Ipr/RA1) (date of deposit: Aug. 21, 2020)

Those 3 strains of mice are similar to, but slightly different from, each other in chromosome nucleic acid sequence, and each develop ankylosis, sialoadenitis, and vasculitis.

The inventors of the present invention have administered an antitumor drug and/or an immune checkpoint inhibitor to each of the 8 strains of MXH recombinant inbred mice (MXH/Mo/Ipr) each showing the disease state of a collagen disease and the 3 strains of McH recombinant congenic mice each developing arthritis, ankylosis, or the like, and have observed the mice. As a result, the inventors have found that an adverse effect or a disease state induced by the antitumor drug and/or the immune checkpoint inhibitor can be assessed.

In this specification, the assessment of an adverse effect and the identification of a chromosome region related to the adverse effect can be performed by a person who performs the respective methods of the present invention such as a person skilled in the art in accordance with the present disclosure without excessive labor.

In some embodiments, the present invention provides a method of assessing an adverse effect induced by an antitumor drug and/or an immune checkpoint inhibitor, the method including:
administering the antitumor drug and/or the immune checkpoint inhibitor to a model mouse that is any one of
(i) one or two or more kinds selected from the group consisting of: an MXH7/Mo/Ipr mouse, an MXH10/Mo/Ipr mouse, an MXH28/Mo/Ipr mouse, an MXH36/Mo/Ipr mouse, an MXH41/Mo/Ipr mouse, an MXH43/Mo/Ipr mouse, an MXH51/Mo/Ipr mouse, and an MXH54/Mo/Ipr mouse, or
(ii) one or two or more kinds selected from the group consisting of: an McH/Mo/Ank.Y^{C3H} mouse, an McH/Mo/Ank.Y^{MRL} mouse, and an McH/Mo/Ipr/RA1 mouse; and
assessing the adverse effect induced by the antitumor drug and/or the immune checkpoint inhibitor.

The term “an antitumor drug and/or an immune checkpoint inhibitor” refers to the antitumor drug or the immune checkpoint inhibitor, or both of the drug and the inhibitor.

Examples of the antitumor drug include an alkylating agent, a platinating agent, an antimetabolite, a topoisomerase inhibitor, a DNA intercalator, an antimitotic agent, an anticancer antibiotic, a plant-derived anticancer agent, an epigenomic drug, an immunomodulator, a molecular targeted drug, an angiogenesis inhibitor, and other antitumor drugs. Those drugs may be used alone or in combination thereof. Those drugs may be appropriately selected by a person skilled in the art in accordance with a tumor of interest.

The “alkylating agent” is not particularly limited, but examples thereof include nitrogen mustard, nitrogen mustard-N-oxide hydrochloride, chlorambucil, cyclophosphamide, ifosfamide, thiotepa, carboquone, improsulfan tosilate, busulfan, nimustine hydrochloride, mitobronitol, melphalan, dacarbazine, procarbazine, ranimustine, estramustine sodium phosphate, triethylenemelamine, carmustine, lomustine, streptozocin, pipobroman, etoglucid, altretamine, ambamustine, dibrospidium hydrochloride, fotemustine, prednimustine, bendamustine, uramustine, semustine, pumitepa, Ribomustin, temozolomide, treosulfan, trofosfamide, zinostatin stimalamer, adozelesin, cystemustine, bizelesin, mechloethamine, uracil mustard, streptozocin, trabectedin, becaterin, chlormethine, mannosulfan, triaziquone, procarbazine, canfosfamide, nitrosourea, and combinations thereof.

The “platinating agent” is not particularly limited, but examples thereof include cisplatin, carboplatin, miboplatin, nedaplatin, satraplatin, oxaliplatin, triplatin tetranitrate, and combinations thereof.

The “antimetabolite” is not particularly limited, but examples thereof include an antifolate, a pyrimidine metabolism inhibitor, a purine metabolism inhibitor, a ribonucleotide reductase inhibitor, and a nucleotide analog.

The “antimetabolite” is not particularly limited, but examples thereof include mercaptopurine, 6-mercaptopurine riboside, thioinosine, methotrexate, pemetrexed, eocitabine, enocitabine, cytarabine, cytarabine ocfosfate, ancitabine hydrochloride, a 5-FU drug (e.g., Fluorouracil, Carzonal, Benton, Lunachol, Lunapon, Tegafur, Tegafur-uracil, Tegafur-gimeracil-oteracil-potassium (TS-1), UFT, Doxifluridine, Carmofur, Galocitabine, Emitefur, or Capecitabine), aminopterin, nelarabine, leucovorin calcium, Tabloid, Butocine, folinate calcium, levofolinate calcium, cladribine, Emitefur, fludarabine, gemcitabine, hydroxycarbamide, pentostatin, piritrexim, idoxuridine, mitoguazone, tiazofurin, ambamustine, bendamustine, floxuridine, nelarabine, leucovorin, hydroxyurea, thioguanine, asparaginase, bortezomib, raltitrexed, clofarabin, enocitabine, sapacitabine, azacitidine, sulfadiazine, sulfamethoxazole, trimethoprim, Liproxstatin-1, D4476, Xanthohumol, Epacadostat (INCB024360), Vidofludimus, P7C3, GMX1778 (CHS828), NCT-501, SW033291, Ro61-8048, and combinations thereof.

The “topoisomerase inhibitor” is not particularly limited, but examples thereof include doxorubicin, daunorubicin, epirubicin, idarubicin, anthracenedione, mitoxantrone, mitomycin C, bleomycin, dactinomycin, plicamycin, irinotecan, camptothecin, rubitecan, belotecan, etoposide, teniposide, topotecan, amsacrine, and combinations thereof.

The “DNA intercalator” is not particularly limited, but examples thereof include proflavin, doxorubicin (adriamycin), daunorubicin, dactinomycin, thalidomide, and combinations thereof.

The “antimitotic agent” is not particularly limited, but examples thereof include paclitaxel, a paclitaxel derivative (e.g., DHA paclitaxel, polyglutamate paclitaxel, nab-paclitaxel, a paclitaxel micelle, 7α-glucosyloxyacetyl paclitaxel, or BMS-275183), docetaxel, vinorelbine, vincristine, vinblastine, vindesine, vinzolidine, etoposide, teniposide, ixabepilone, larotaxel, ortataxel, tesetaxel, ispinesib, colchicine, vinflunine, and combinations thereof.

The “anticancer antibiotic” is not particularly limited, but examples thereof include actinomycin D, actinomycin C, mitomycin C, chromomycin A3, mithramycin A, bleomycin hydrochloride, bleomycin sulfate, peplomycin sulfate, daunorubicin hydrochloride, doxorubicin hydrochloride, aclarubicin hydrochloride, pirarubicin hydrochloride, epirubicin hydrochloride, amrubicin hydrochloride, neocarzinostatin, zinostatin stimalamer, mithramycin, sarkomycin, carcinophilin, mitotane, zorubicin hydrochloride, mitoxantrone hydrochloride, idarubicin hydrochloride, liposomal doxorubicin, and combinations thereof.

The “plant-derived anticancer agent” is not particularly limited, but examples thereof include irinotecan, nogitecan, etoposide, etoposide phosphate, eribulin, sobuzoxane, vinblastine sulfate, vincristine sulfate, vindesine sulfate, teniposide, paclitaxel, a paclitaxel injection, docetaxel, DJ-927, vinorelbine, topotecan, and combinations thereof.

The “epigenomic drug” is not particularly limited, but examples thereof include a DNA methylation inhibitor, a histone deacetylase (HDAC) inhibitor, a DNA methyltransferase (DNMT) inhibitor, a histone deacetylase activator, a histone demethylase inhibitor, and a methylated nucleotide.

The “epigenomic drug” is not particularly limited, but specific examples thereof include vorinostat, belinostat, mocetinostat (MGCD0103), entinostat (SNDX-275), romidepsin, azacytidine, decitabine, GSK2879552 2HI, SGC707, ORY-1001 (RG-6016), PFI-4, SirReal2, GSK2801, CPI-360, GSK503, AMI-1, CPI-169, and combinations thereof.

The “immunomodulator” is not particularly limited, but examples thereof include thalidomide, lenalidomide, pomalidomide, and combinations thereof.

The “molecular targeted drug” may be a low molecular weight compound or an antibody. The “molecular targeted drug” is not particularly limited, but examples thereof include a kinase inhibitor, a proteasome inhibitor, a monoclonal antibody, an mTOR inhibitor, a TNF inhibitor, and a T-cell inhibitor.

The “kinase inhibitor” is not particularly limited, but examples thereof include a tyrosine kinase inhibitor, a serine/threonine kinase inhibitor, a Raf kinase inhibitor, a cyclin-dependent kinase (CDK) inhibitor, and a mitogen-activated protein kinase (MEK) inhibitor.

The “kinase inhibitor” is not particularly limited, but specific examples thereof include imatinib, gefitinib, erlotinib, afatinib, dasatinib, bosutinib, vandetanib, sunitinib, axitinib, pazopanib, lenvatinib, lapatinib, nintedanib, nilotinib, crizotinib, ceritinib, alectinib, ruxolitinib, tofacitinib, ibrutinib, sorafenib, vemurafenib, dabrafenib, palbociclib, trametinib, regorafenib, cediranib, lestaurtinib, vandetanib, vatalanib, seliciclib, tivantinib, canertinib, pelitinib, tesevatinib, cediranib, motesanib, midostaurin, foretinib, cabozantinib, selumetinib, neratinib, volasertib, saracatinib, enzastaurin, tandutinib, semaxanib, alvocidib, ICR62, AEE788, PD035901, PD1535, TK787, BBI503, E6201, E7050, and combinations thereof.

The “proteasome inhibitor” is not particularly limited, but examples thereof include bortezomib, carfilzomib, and combinations thereof.

The “monoclonal antibody” is not particularly limited, but examples thereof include an anti-CD22 antibody, an anti-CD20 antibody, an anti-CD25 antibody, an anti-CD30 antibody, an anti-CD33 antibody, an anti-CD5 antibody, an anti-CD52 antibody, an anti-epidermal growth factor receptor antibody (EGFR antibody), an anti-vascular endothelial growth factor antibody (VEGF antibody), an anti-TNF-α antibody, an anti-IL-1 receptor antibody, an anti-IL-2 receptor antibody, an anti-IL-5 receptor antibody, an anti-IL-6 receptor antibody, an anti-HER2 antibody, an anti-IgE antibody, an anti-IgG antibody, an anti-RS virus antibody, an anti-CCR4 antibody, an anti-CTLA-4 (cytotoxic T lymphocyte-associated antigen 4, CD152) antibody, an anti-PD-1 antibody, an anti-PD-L1 antibody, an anti-receptor activator of nuclear factor κB ligand (RANKL) antibody, an anti-c-Met antibody, and an anti-CXCR4 antibody. Those antibodies may be mouse antibodies, human antibodies, or chimeric antibodies thereof.

The “monoclonal antibody” is not particularly limited, but specific examples thereof include ibritumomab tiuxetan, rituximab, cetuximab, infliximab, basiliximab, brentuximab vedotin, tocilizumab, trastuzumab, bevacizumab, omalizumab, mepolizumab, gemtuzumab, ozogamicin, palivizumab, ranibizumab, certolizumab, ocrelizumab, mogamulizumab, eculizumab, pertuzumab, alemtuzumab, inotuzumab, panitumumab, ofatumumab, golimumab, adalimumab, ramucirumab, nivolumab, anakinra, denosumab, ipilimumab, pembrolizumab, matuzumab, farletuzumab, MORAb-004, MORAb-009, and combinations thereof.

The “mTOR inhibitor” is not particularly limited, but examples thereof include everolimus (RAD001), rapamycin (sirolimus), AZD8055, temsirolimus (CCI-779, NSC 68364) KU-0063794, Voxtalisib (XL765, SAR245409), MHY1485, dactolisib (BEZ235), PI-103, Torkinib (PP242), ridaforolimus (deforolimus, MK-8669), INK-128 (MLN0128), Torin 1, omiparisib (GSK2126458, GSK458), OSI-027, PF-04691502, apitolisib (GDC-0980, RG7422), GSK1059615, gedatricib (PF-05212384, PKI-587), WYE-132, PP121, WYE-354, AZD2014, Torin 2, WYE-687, CH5132799, WAY-600, ETP-46464, GDC-0349, XL388, zotarolimus (ABT-578), tacrolimus (FK506), BGT226 (NVP-BGT226), palomid 529 (P529), chrysophanic acid, and combinations thereof.

The “TNF inhibitor” is not particularly limited, but examples thereof include etanercept, lenalidomide (CC-5013), pomalidomide, thalidomide, necrostatin-1, and QNZ (EVP4593).

The “T cell inhibitor” is not particularly limited, but an example thereof is abatacept.

The “angiogenesis inhibitor” is not particularly limited, but examples thereof include CM101, IFN-α, IL-12, platelet factor-4, suramin, semaxanib, thrombospondin, a VEGFR antagonist, an angiogenesis inhibitor steroid plus heparin, a cartilage-derived angiogenesis inhibitory factor, a matrix metalloprotease inhibitor, batimastat, marimastat, angiostatin, endostatin, 2-methoxyestradiol, tecogalan, thrombospondin, an αVβ3A inhibitor, linomide, ADH-1, E7820, and combinations thereof.

The “other chemotherapeutic agent” is not particularly limited, but examples thereof include finasteride, sobuzoxane, obatoclax, efaproxiral, tipifarnib, and lonafamib.

The “immune checkpoint inhibitor” is not particularly limited, but examples thereof include inhibitors for PD-1, PD-L1, PD-L2, B7-H3, B7-H4, B7-H5, BTLA, CD80, CD86, CD96, CD47, CD155, CTLA-4, LAG-3, TIGIT, TIM-3, CD111, DNAM-1, Galectin-9, HVEM, Nectin-2, Nectin-3, PVRIG, SIRP alpha, SIRP alpha V2, SIRP gamma, and CD160. The “immune checkpoint inhibitor” may be an antibody against each of those immune checkpoint proteins. Preferred examples of the immune checkpoint inhibitor include immune checkpoint inhibitors selected from the group consisting of: an anti-CTLA-4 antibody; an anti-PD-1 antibody; and an anti-PD-L1 antibody.

In a specific preferred embodiment, the antitumor drug and/or the immune checkpoint inhibitor is the immune checkpoint inhibitor.

Examples of the route of administration of the antitumor drug and/or the immune checkpoint inhibitor to the model mouse include, but not limited to, oral administration, intravenous administration, intralymphatic administration, intraperitoneal administration, transdermal administration, intrathecal administration, intramuscular administration, intranasal administration, transmucosal administration, subcutaneous administration, transrectal administration, and intratumoral administration. Preferred examples of the route of administration include intravenous administration, intralymphatic administration, intraperitoneal administration, and intratumoral administration.

The dose of the antitumor drug and/or the immune checkpoint inhibitor may be appropriately selected by a person skilled in the art in accordance with the kind of drug. Examples of the dose with respect to the body weight of the model mouse include, but not limited to, from 1 μg/kg to 1,000 mg/kg or from about 1 μg/kg to about 1,000 mg/kg, from 1 μg/kg to 100 μg/kg, from 100 μg/kg to 500 μg/kg, from 500 μg/kg to 1,000 μg/kg, from 1 mg/kg to 10 mg/kg, from 10 mg/kg to 100 mg/kg, from 100 mg/kg to 500 mg/kg, from 200 mg/kg to 300 mg/kg, from 100 mg/kg to 250 mg/kg, from 200 mg/kg to 400 mg/kg, from 250 mg/kg to 500 mg/kg, from 250 mg/kg to 750 mg/kg, from 50 mg/kg to 750 mg/kg, from 1 mg/kg to 10 mg/kg, and from 100 mg/kg to 1,000 mg/kg.

Examples of the adverse effect include, but not limited to, side effects, such as weight loss and increased lymph node weight, interstitial pneumonia, sialoadenitis, vasculitis, arthritis, glomerulonephritis, dacryoadenitis, colitis, hypothyroidism, liver damage, a rash, hypophysitis, diabetes, peripheral neuropathy, myasthenia gravis, and a combination of two or more thereof.

In a specific preferred embodiment, the adverse effect is an immune-related adverse effect (irAE). The immune-related adverse effect includes interstitial pneumonia, sialoadenitis, vasculitis, arthritis, glomerulonephritis, dacryoadenitis, or a combination of two or more thereof.

In a specific embodiment, the assessment of the adverse effect induced by the antitumor drug and/or the immune checkpoint inhibitor includes determining the presence or absence of the adverse effect from the state of a tissue or a cell in the model mouse having administered thereto the antitumor drug and/or the immune checkpoint inhibitor. Examples of the state of the tissue or the cell include the disease state of the tissue, the form of the cell, and the presence or absence of the expression of a specific molecule related to the adverse effect in the cell.

The presence or absence of the adverse effect may be determined by, for example, comparing the state of the tissue or the cell in the model mouse before the administration of the antitumor drug and/or the immune checkpoint inhibitor, and the state of the tissue or the cell in the same model mouse after the administration of the antitumor drug and/or the immune checkpoint inhibitor to each other. Alternatively, the presence or absence of the adverse effect may be determined by comparing the state of the tissue or the cell in the model mouse to which the antitumor drug and/or the immune checkpoint inhibitor is not administered, and the state of the tissue or the cell in the model mouse having administered thereto the antitumor drug and/or the immune checkpoint inhibitor to each other. Alternatively, the presence or absence of the adverse effect may be determined by comparing the state of the tissue or the cell in the model mouse having administered thereto a pharmaceutically acceptable carrier, which is free of the antitumor drug and/or the immune checkpoint inhibitor, and the state of the tissue or the cell in the model mouse having administered thereto the antitumor drug and/or the immune checkpoint inhibitor to each other.

In a specific embodiment, the assessment of the adverse effect induced by the antitumor drug and/or the immune checkpoint inhibitor includes determining the presence or absence of a difference in adverse effect from the state of a tissue or a cell in the same, or same kind of, model mouse having administered thereto two or more kinds of different drugs. Examples of the state of the tissue or the cell include the disease state of the tissue, the form of the cell, and the presence or absence of the expression of a specific molecule related to the adverse effect in the cell.

For example, when the states of the tissues or the cells in the same, or same kind of, model mouse having administered thereto the two or more kinds of different drugs are different from each other, it can be determined that the degree of the adverse effect induced in such model mouse varies depending on the drugs. When the states of the tissues or the cells in the same, or same kind of, model mouse having administered thereto the two or more kinds of different drugs are similar or identical to each other, or are not significantly different from each other, it can be determined that the degrees of the adverse effect induced in such model mouse are similar or identical to each other, or are not significantly different from each other irrespective of the drugs.

The above-mentioned method may further include inoculating a tumor to the above-mentioned model mouse before the step of administering the antitumor drug and/or the immune checkpoint inhibitor. The inoculation of the tumor to the model mouse can produce a model mouse suffering from the tumor.

Examples of the kind of the tumor to be inoculated include, but not limited to, an MRL/N1 cell (PLos ONE 8 (2): e55797.) and an FM3A cell (J Immunol Methods. 2013 Mar. 29; 389 (1-2): 69-78), and the tumor may be appropriately selected by a person skilled in the art. Examples of the site to which the tumor is inoculated include, but not limited to, a lymph node and a flank, and the site may be appropriately selected by a person skilled in the art.

The above-mentioned method may further include assessing the state of the tumor in the model mouse after the step of administering the antitumor drug and/or the immune checkpoint inhibitor. When the tumor in the model mouse is suppressed, and no adverse effect is present, it can be determined that the antitumor drug and/or the immune checkpoint inhibitor is effective in treating the tumor in the model mouse. When the tumor in the model mouse is suppressed, and an adverse effect is present, it can be determined that the antitumor drug and/or the immune checkpoint inhibitor is not effective in treating the tumor. When the tumor in the model mouse is not suppressed, it can be determined that the antitumor drug and/or the immune checkpoint inhibitor is not effective in treating the tumor.

According to the method of assessing an adverse effect induced by an antitumor drug and/or an immune checkpoint inhibitor according to the above-mentioned embodiment, the adverse effect induced by the antitumor drug and/or the immune checkpoint inhibitor can be simply assessed by using a disease model mouse that is an MXH recombinant inbred mouse or an McH congenic mouse.

In some embodiments, the present invention provides a method of screening an antitumor drug and/or an immune checkpoint inhibitor, the method including:
administering a test substance that is the antitumor drug and/or the immune checkpoint inhibitor to a model mouse that is any one of
(i) one or two or more kinds selected from the group consisting of: an MXH7/Mo/Ipr mouse, an MXH10/Mo/Ipr mouse, an MXH28/Mo/Ipr mouse, an MXH36/Mo/Ipr mouse, an MXH41/Mo/Ipr mouse, an MXH43/Mo/Ipr mouse, an MXH51/Mo/Ipr mouse, and an MXH54/Mo/Ipr mouse, or
(ii) one or two or more kinds selected from the group consisting of: an McH/Mo/Ank.Y^{C3H} mouse, an McH/Mo/Ank.Y^{MRL} mouse, and an McH/Mo/Ipr/RA1 mouse; and
assessing the presence or absence of an adverse effect in the model mouse after the administration of the test substance.

Examples of the antitumor drug include an alkylating agent, a platinating agent, an antimetabolite, a topoisomerase inhibitor, a DNA intercalator, an antimitotic agent, an anticancer antibiotic, a plant-derived anticancer agent, an epigenomic drug, an immunomodulator, a molecular targeted drug, an angiogenesis inhibitor, and other antitumor drugs. Those drugs may be used alone or in combination thereof. Those drugs may be appropriately selected by a person skilled in the art in accordance with a tumor of interest.

Examples of the immune checkpoint inhibitor include, but not limited to, inhibitors for PD-1, PD-L1, PD-L2, B7-H3, B7-H4, B7-H5, BTLA, CD80, CD86, CD96, CD47, CD155, CTLA-4, LAG-3, TIGIT, TIM-3, CD111, DNAM-1, Galectin-9, HVEM, Nectin-2, Nectin-3, PVRIG, SIRP alpha, SIRP alpha V2, SIRP gamma, and CD160. The immune checkpoint inhibitor may be an antibody against each of those immune checkpoint proteins. Preferred examples of the immune checkpoint inhibitor include immune checkpoint inhibitors selected from the group consisting of: an anti-CTLA-4 antibody; an anti-PD-1 antibody; and an anti-PD-L1 antibody.

In a specific preferred embodiment, the test substance is an immune checkpoint inhibitor.

Examples of the route of administration of the test substance to the model mouse include, but not limited to, oral administration, intravenous administration, intralymphatic administration, intraperitoneal administration, transdermal administration, intrathecal administration, intramuscular administration, intranasal administration, transmucosal administration, subcutaneous administration, and transrectal administration. Preferred examples of the route of administration include intravenous administration, intralymphatic administration, and intraperitoneal administration.

The dose of the test substance may be appropriately selected by a person skilled in the art in accordance with the kind of drug. Examples of the dose with respect to the body weight of the model mouse include, but not limited to, from 1 μg/kg to 1,000 mg/kg or from about 1 μg/kg to about 1,000 mg/kg, from 1 μg/kg to 100 μg/kg, from 100 g/kg to 500 μg/kg, from 500 μg/kg to 1,000 μg/kg, from 1 mg/kg to 10 mg/kg, from 10 mg/kg to 100 mg/kg, from 100 mg/kg to 500 mg/kg, from 200 mg/kg to 300 mg/kg, from 100 mg/kg to 250 mg/kg, from 200 mg/kg to 400 mg/kg, from 250 mg/kg to 500 mg/kg, from 250 mg/kg to 750 mg/kg, from 50 mg/kg to 750 mg/kg, from 1 mg/kg to 10 mg/kg, and from 100 mg/kg to 1,000 mg/kg.

Examples of the adverse effect include, but not limited to, side effects, such as weight loss and increased lymph node weight, interstitial pneumonia, sialoadenitis, vasculitis, arthritis, glomerulonephritis, dacryoadenitis, colitis, hypothyroidism, liver damage, a rash, hypophysitis, diabetes, peripheral neuropathy, myasthenia gravis, and a combination of two or more thereof.

In a specific preferred embodiment, the adverse effect is an immune-related adverse effect (irAE). The immune-related adverse effect includes interstitial pneumonia, sialoadenitis, vasculitis, arthritis, glomerulonephritis, dacryoadenitis, or a combination of two or more thereof.

In a specific embodiment, the assessment of the presence or absence of the adverse effect in the model mouse after the administration of the test substance includes determining the presence or absence of the adverse effect from the state of a tissue or a cell in the model mouse having administered thereto the test substance. Examples of the state of the tissue or the cell include the disease state of the tissue, the form of the cell, and the presence or absence of the expression of a specific molecule related to the adverse effect in the cell.

The presence or absence of the adverse effect may be determined by, for example, comparing the state of the tissue or the cell in the model mouse before the administration of the test substance, and the state of the tissue or the cell in the same model mouse after the administration of the test substance to each other. Alternatively, the presence or absence of the adverse effect may be determined by comparing the state of the tissue or the cell in the model mouse to which the test substance is not administered, and the state of the tissue or the cell in the model mouse having administered thereto the test substance to each other. Alternatively, the presence or absence of the adverse effect may be determined by comparing the state of the tissue or the cell in the model mouse having administered thereto a pharmaceutically acceptable carrier, which is free of the test substance, and the state of the tissue or the cell in the model mouse having administered thereto the test substance to each other.

In a specific embodiment, the assessment of the adverse effect induced by the test substance includes determining the presence or absence of a difference in adverse effect from the state of a tissue or a cell in the same, or same kind of, model mouse having administered thereto two or more kinds of test substances. Examples of the state of the tissue or the cell include the disease state of the tissue, the form of the cell, and the presence or absence of the expression of a specific molecule related to the adverse effect in the cell.

For example, when the states of the tissues or the cells in the same, or same kind of, model mouse having administered thereto the two or more kinds of test substances are different from each other, it can be determined that the degree of the adverse effect induced in such model mouse varies in accordance with the test substances. When the states of the tissues or the cells in the same, or same kind of, model mouse having administered thereto the two or more kinds of test substances are similar or identical to each other, or are not significantly different from each other, it can be determined that the degrees of the adverse effect induced in such model mouse are similar or identical to each other, or are not significantly different from each other irrespective of the test substances.

The above-mentioned method may further include inoculating a tumor to the above-mentioned model mouse before the step of administering the test substance. The inoculation of the tumor to the model mouse can produce a model mouse suffering from the tumor.

Examples of the kind of the tumor to be inoculated include, but not limited to, an MRL/N1 cell (PLos ONE 8 (2): e55797.) and an FM3A cell (J Immunol Methods. 2013 Mar. 29; 389 (1-2): 69-78), and the tumor may be appropriately selected by a person skilled in the art. Examples of the site to which the tumor is inoculated include, but not limited to, a lymph node and a flank, and the site may be appropriately selected by a person skilled in the art.

The above-mentioned method may further include assessing the state of the tumor in the model mouse after the step of administering the test substance. When the tumor in the model mouse is suppressed, and no adverse effect is present, it can be determined that the test substance is effective in treating the tumor in the model mouse. When the tumor in the model mouse is suppressed, and an adverse effect is present, it can be determined that the test substance is not effective in treating the tumor. When the tumor in the model mouse is not suppressed, it can be determined that the test substance is not effective in treating the tumor.

The above-mentioned method may further include the steps of: administering a plurality of test substances; and selecting a test substance, which has a larger tumor-suppressing effect and is free of any adverse effect, from the plurality of test substances. According to such method, a test substance that is more suitable for the treatment can be simply screened from the plurality of test substances.

According to the method of screening an antitumor drug and/or an immune checkpoint inhibitor as described above, the antitumor drug and/or the immune checkpoint inhibitor that is free of any adverse effect can be simply selected by using a disease model mouse that is an MXH recombinant inbred mouse or an McH congenic mouse.

In another embodiment, the present invention provides a method of screening an antitumor drug and/or an immune checkpoint inhibitor that is free of any adverse effect or is reduced in adverse effect, the method including:
administering a test substance that is the antitumor drug and/or the immune checkpoint inhibitor to a model mouse that is any one of
(i) one or two or more kinds selected from the group consisting of: an MXH7/Mo/Ipr mouse, an MXH10/Mo/Ipr mouse, an MXH28/Mo/Ipr mouse, an MXH36/Mo/Ipr mouse, an MXH41/Mo/Ipr mouse, an MXH43/Mo/Ipr mouse, an MXH51/Mo/Ipr mouse, and an MXH54/Mo/Ipr mouse, or
(ii) one or two or more kinds selected from the group consisting of: an McH/Mo/Ank.Y^{C3H} mouse, an McH/Mo/Ank.Y^{MRL} mouse, and an McH/Mo/Ipr/RA1 mouse, the administration being performed for each of a plurality of test substances;
assessing the presence or absence of an adverse effect in the model mouse after the administration of each of the plurality of test substances; and
selecting a test compound that is free of any adverse effect or is reduced in adverse effect from the plurality of test substances.

Examples of the antitumor drug include an alkylating agent, a platinating agent, an antimetabolite, a topoisomerase inhibitor, a DNA intercalator, an antimitotic agent, an anticancer antibiotic, a plant-derived anticancer agent, an epigenomic drug, an immunomodulator, a molecular targeted drug, an angiogenesis inhibitor, and other antitumor drugs. Those drugs may be used alone or in combination thereof. Those drugs may be appropriately selected by a person skilled in the art in accordance with a tumor of interest.

Examples of the immune checkpoint inhibitor include, but not limited to, inhibitors for PD-1, PD-L1, PD-L2, B7-H3, B7-H4, B7-H5, BTLA, CD80, CD86, CD96, CD47, CD155, CTLA-4, LAG-3, TIGIT, TIM-3, CD111, DNAM-1, Galectin-9, HVEM, Nectin-2, Nectin-3, PVRIG, SIRP alpha, SIRP alpha V2, SIRP gamma, and CD160. The immune checkpoint inhibitor may be an antibody against each of those immune checkpoint proteins. Preferred examples of the immune checkpoint inhibitor include immune checkpoint inhibitors selected from the group consisting of: an anti-CTLA-4 antibody; an anti-PD-1 antibody; and an anti-PD-L1 antibody.

In a specific preferred embodiment, the test substance is an immune checkpoint inhibitor.

Examples of the route of administration of the test substance to the model mouse include, but not limited to, oral administration, intravenous administration, intralymphatic administration, intraperitoneal administration, transdermal administration, intrathecal administration, intramuscular administration, intranasal administration, transmucosal administration, subcutaneous administration, and transrectal administration. Preferred examples of the route of administration include intravenous administration, intralymphatic administration, and intraperitoneal administration.

The dose of the test substance may be appropriately selected by a person skilled in the art in accordance with the kind of drug. Examples of the dose with respect to the body weight of the model mouse include, but not limited to, from 1 μg/kg to 1,000 mg/kg or from about 1 μg/kg to about 1,000 mg/kg, from 1 μg/kg to 100 μg/kg, from 100 μg/kg to 500 μg/kg, from 500 μg/kg to 1,000 μg/kg, from 1 mg/kg to 10 mg/kg, from 10 mg/kg to 100 mg/kg, from 100 mg/kg to 500 mg/kg, from 200 mg/kg to 300 mg/kg, from 100 mg/kg to 250 mg/kg, from 200 mg/kg to 400 mg/kg, from 250 mg/kg to 500 mg/kg, from 250 mg/kg to 750 mg/kg, from 50 mg/kg to 750 mg/kg, from 1 mg/kg to 10 mg/kg, and from 100 mg/kg to 1,000 mg/kg.

Examples of the adverse effect include, but not limited to, side effects, such as weight loss and increased lymph node weight, interstitial pneumonia, sialoadenitis, vasculitis, arthritis, glomerulonephritis, dacryoadenitis, colitis, hypothyroidism, liver damage, a rash, hypophysitis, diabetes, peripheral neuropathy, myasthenia gravis, and a combination of two or more thereof.

The above-mentioned method may further include inoculating a tumor to the above-mentioned model mouse before the step of administering the test substance. The inoculation of the tumor to the model mouse can produce a model mouse suffering from the tumor.

Examples of the kind of the tumor to be inoculated include, but not limited to, an MRL/N1 cell (PLos ONE 8 (2): e55797.) and an FM3A cell (J Immunol Methods. 2013 Mar. 29; 389(1-2): 69-78), and the tumor may be appropriately selected by a person skilled in the art. Examples of the site to which the tumor is inoculated include, but not limited to, a lymph node and a flank, and the site may be appropriately selected by a person skilled in the art.

The above-mentioned method may further include assessing the state of the tumor in the model mouse after the step of administering the test substance. The assessment of the state of the tumor in the model mouse includes determining whether or not the tumor in the model mouse is suppressed.

The selection of the test compound that is free of any adverse effect or is reduced in adverse effect from the plurality of test substances may include a step of selecting the test substance, which has a larger tumor-suppressing effect and is further reduced in adverse effect, from the plurality of test substances, a step of selecting the test substance, which has a larger tumor-suppressing effect and is free of any adverse effect, therefrom, or a step of selecting the test substance, which has the same level of tumor-suppressing effect and is further reduced in adverse effect, therefrom. According to such method, a test substance that is more suitable for the treatment of the tumor can be simply screened from the plurality of test substances.

According to the above-mentioned method of screening an antitumor drug and/or an immune checkpoint inhibitor, the antitumor drug and/or the immune checkpoint inhibitor that is free of any adverse effect or is reduced in adverse effect can be simply selected by using a disease model mouse that is an MXH recombinant inbred mouse or an McH congenic mouse.

In some embodiments, the present invention provides a method of identifying a chromosome region related to an adverse effect induced by administration of an antitumor drug and/or an immunotherapy drug, the method including;
administering the antitumor drug and/or the immunotherapy drug to a model mouse is any one of
(i) one or two or more kinds selected from the group consisting of: an MXH7/Mo/Ipr mouse, an MXH10/Mo/Ipr mouse, an MXH28/Mo/Ipr mouse, an MXH36/Mo/Ipr mouse, an MXH41/Mo/Ipr mouse, an MXH43/Mo/Ipr mouse, an MXH51/Mo/Ipr mouse, and an MXH54/Mo/Ipr mouse, or
(ii) one or two or more kinds selected from the group consisting of: an McH/Mo/Ank.Y^{C3H} mouse, an McH/Mo/Ank.Y^{MRL} mouse, and an McH/Mo/Ipr/RA1 mouse; and
identifying the chromosome region related to the adverse effect induced by the administration of the antitumor drug and/or the immunotherapy drug.

The chromosome region may be a gene region, or may be a nucleic acid region except a gene.

Examples of the antitumor drug include an alkylating agent, a platinating agent, an antimetabolite, a topoisomerase inhibitor, a DNA intercalator, an antimitotic agent, an anticancer antibiotic, a plant-derived anticancer agent, an epigenomic drug, an immunomodulator, a molecular targeted drug, an angiogenesis inhibitor, and other antitumor drugs. Those drugs may be used alone or in combination thereof. Those drugs may be appropriately selected by a person skilled in the art in accordance with a tumor of interest.

Examples of the immunotherapy drug include picibanil, krestin, schizophyllan, lentinan, ubenimex, interferon (IL)-α, interferon (IL)-β, interferon (IL)-γ, interleukin, a macrophage colony stimulating factor, a granulocyte colony stimulating factor, erythropoietin, lymphotoxin, a BCG vaccine, Corynebacterium parvum, levamisole, polysaccharide K, procodazole, an anti-CTLA-4 antibody, an anti-PD-1 antibody, an anti-PD-L1 antibody, and a TLR agonist (e.g., a TLR7 agonist, a TLR8 agonist, or a TLR9 agonist). Those antibodies may be mouse antibodies, human antibodies, or chimeric antibodies thereof.

In a specific preferred embodiment, the antitumor drug and/or the immunotherapy drug is the immune checkpoint inhibitor. Examples of the immune checkpoint inhibitor include, but not limited to, inhibitors for PD-1, PD-L1, PD-L2, B7-H3, B7-H4, B7-H5, BTLA, CD80, CD86, CD96, CD47, CD155, CTLA-4, LAG-3, TIGIT, TIM-3, CD111, DNAM-1, Galectin-9, HVEM, Nectin-2, Nectin-3, PVRIG, SIRP alpha, SIRP alpha V2, SIRP gamma, and CD160. The “immune checkpoint inhibitor” may be an antibody against each of those immune checkpoint proteins. Preferred examples of the immune checkpoint inhibitor include immune checkpoint inhibitors selected from the group consisting of: an anti-CTLA-4 antibody; an anti-PD-1 antibody; and an anti-PD-L1 antibody.

Examples of the route of administration of the antitumor drug and/or the immunotherapy drug to the model mouse include, but not limited to, oral administration, intravenous administration, intralymphatic administration, intraperitoneal administration, transdermal administration, intrathecal administration, intramuscular administration, intranasal administration, transmucosal administration, subcutaneous administration, and transrectal administration. Preferred examples of the route of administration include intravenous administration, intralymphatic administration, and intraperitoneal administration.

The dose of the antitumor drug and/or the immunotherapy drug may be appropriately selected by a person skilled in the art in accordance with the kind of drug. Examples of the dose with respect to the body weight of the model mouse include, but not limited to, from 1 μg/kg to 1,000 mg/kg or from about 1 μg/kg to about 1,000 mg/kg, from 1 μg/kg to 100 μg/kg, from 100 μg/kg to 500 μg/kg, from 500 μg/kg to 1,000 μg/kg, from 1 mg/kg to 10 mg/kg, from 10 mg/kg to 100 mg/kg, from 100 mg/kg to 500 mg/kg, from 200 mg/kg to 300 mg/kg, from 100 mg/kg to 250 mg/kg, from 200 mg/kg to 400 mg/kg, from 250 mg/kg to 500 mg/kg, from 250 mg/kg to 750 mg/kg, from 50 mg/kg to 750 mg/kg, from 1 mg/kg to 10 mg/kg, and from 100 mg/kg to 1,000 mg/kg.

Examples of the adverse effect include, but not limited to, side effects, such as weight loss and increased lymph node weight, interstitial pneumonia, sialoadenitis, vasculitis, arthritis, glomerulonephritis, dacryoadenitis, colitis, hypothyroidism, liver damage, a rash, hypophysitis, diabetes, peripheral neuropathy, myasthenia gravis, and a combination of two or more thereof.

In a specific preferred embodiment, the adverse effect is an immune-related adverse effect (irAE). The immune-related adverse effect includes interstitial pneumonia, sialoadenitis, vasculitis, arthritis, glomerulonephritis, dacryoadenitis, or a combination of two or more thereof.

In a specific embodiment, the identification of the chromosome region related to the adverse effect induced by the administration of the antitumor drug and/or the immune checkpoint inhibitor includes identifying the chromosome region related to the adverse effect induced by the administration of the antitumor drug and/or the immune checkpoint inhibitor from the list of the chromosome regions of the model mouse related to the adverse effect induced by the administration of the antitumor drug and/or the immune checkpoint inhibitor.

As shown in Table 1, some chromosome regions related to the development of sialoadenitis, vasculitis, arthritis, glomerulonephritis, and dacryoadenitis are known.

For example, when an adverse effect is determined from the state of a tissue or a cell in the model mouse having administered thereto the antitumor drug and/or the immune checkpoint inhibitor, and the adverse effect is glomerulonephritis, a chromosome region related to the adverse effect can be identified as the Agnm1 gene of a fourth chromosome, the Agnm2 gene of the fourth chromosome, or the Agnm3 gene of a fifth chromosome.

In addition, when the same antitumor drug and/or immune checkpoint inhibitor is administered to each of a plurality of kinds of, in other words, two or more kinds of model mice having different genotypes, and adverse effects induced by the administration of the same antitumor drug and/or immune checkpoint inhibitor are different from each other (e.g., there is a difference in presence or absence of adverse effect induction between the plurality of kinds of model mice), a chromosome region related to an adverse effect can be identified with higher accuracy.

TABLE 1
Table of diseases and disease-sensitive gene loci in MRL/Ipr mouse

| Lesion | Symbol | Name | ID | Chromosome | Position | References |
|---|---|---|---|---|---|---|
| Sialoadenitis | Asm1 | autoimmune sialoadenitis | MGI: | 10 | 39.0 cM | Nishihara et al. Arthritis Rheum |
| | | in MRL mice 1 | 2150642 | | | 1999: 42: 2616 |
| Slaloadenitis | Asm2 | autoimmune sialoadenitis | MGI: | 4 | 51.0 cM | Nishihara et al. Arthritis Rheum |
| | | in MRL mice 2 | 2150643 | | | 1999; 42: 2616 |
| Sialoadenitis | Asm3 | autoimmune sialoadenitis | MGI: | 1 | 51.0 cM | Kamo et al. Invest |
| | | in MRL mice 3 | 4410443 | | | Ophthalmol Vis Sci 2009; 50: 3257 |
| Sialoadenitis | Asm4 | autoimmune sialoadenitis | MGI: | 2 | 51.0 cM | Kamo et al. Invest |
| | | in MRL mice 4 | 4410444 | | | Ophthalmol Vis Sci 2009; 50: 3257 |
| Sialoadenitis | Asm5 | autoimmune sialoadenitis | MGI: | 2 | 51.0 cM | Kamo et al. Invest |
| | | in MRL mice 5 | 4410446 | | | Ophthalmol Vis Sci 2009; 50: 3257 |
| Vasculitis | Arvm1 | autoimmune renal | MGI: | 4 | 19.8 cM | Qu et al. Eur J Immunol 2000; 30: |
| | | vasculitis in MRL mice 1 | 2149546 | | | 2027 |
| Vasculitis | Arvm2 | autoimmune renal | MGI: | 4 | 58.0 cM | Qu et al. Eur J Immunol 2000; 30: |
| | | vasculitis in MRL mice 2 | 2149547 | | | 2027 |
| Vasculitis | Aaom1 | autoimmune aortitis in | MGI: | 4 | 13.3 cM | Yamada et al. Arthritis Rheum |
| | | MRL mice 1 | 2680905 | | | 2003; 48: 1445 |
| Vasculitis | Aevm1 | autoimmune extremity | MGI: | 8 | 33.0 cM | Yamada et al. Arthritis Rheum |
| | | vasculitis in MRL mice 1 | 2680906 | | | 2003: 48: 1445 |
| Vasculitis | Aevm2 | autoimmune extremity | MGI: | 5 | 65.0 cM | Yamada et al. Arthritis Rheum |
| | | vasculitis in MRL mice 2 | 2680907 | | | 2003; 48: 1445 |
| Arthritis | Paam1 | progression of autoimmune | MGI: | 15 | 18.0 cM | Kamogawa et al. Arthritis Rheum |
| | | arthritis in MRL mice 1 | 2387302 | | | 2002; 46: 1067 |
| Arthritis | Paam2 | progression of autoimmune | MGI: | 19 | 49.0 cM | Kamogawa et al. Arthritis Rheum |
| | | arthritis in MRL mice 2 | 2387303 | | | 2002; 46: 1067 |
| Arthritis | Artmd1 | arthropathy In MRL and | MGI: | 10 | 40.0 cM | Oishi et al. Arthritis Rheum 2005; |
| | (Amd1) | DBA/1 mice 1 | 3588383 | | | 52: 959 |
| Arthritis | Artmd2 | arthropathy in MRL and | MGI: | 3 | 29.5 cM | Oishi et al. Arthritis Rheum 2005; |
| | (Amd2) | DBA/1 mice 2 | 3588384 | | | 52: 959 |
| Glomerulonephritis | Agnm1 | autoimmune glomerulonephritis | MGI: | 4 | 22.0 cM | Miyazaki et al. Eur J Immunol |
| | | in MRL mice | 3582415 | | | 2005: 35: 1510 |
| Glomerulonephritis | Agnm2 | autoimmune glomerulonephritis | MGI: | 4 | 53.0 cM | Miyazaki et al. Eur J Immunol |
| | | in MRL mice 2 | 3582416 | | | 2005; 35: 1510 |
| Glomerulonephritis | Agnm3 | autoimmune glomerulonephritis | MGI: | 5 | 56.0 cM | Miyazaki et al. Eur J Immunol |
| | | in MRL mice 3 | 3582417 | | | 2005; 35: 1510 |
| Dacryoadenitis | Adacm1 | autoimmune dacryoadenitisin | MGI: | 1 | 64.1 cM | Kamo et al. Invest |
| | | MRL mice 1 | 4410449 | | | Ophthalmol Vis Sci 2009; 50: 3257 |
| Dacryoadenitis | Adacm2 | autoimmune dacryoadenitisin | MGI: | 2 | 88.4 cM | Kamo et al. Invest |
| | | MRL mice 1 | 4410450 | | | Ophthalmol Vis Sci 2009; 50: 3257 |
| Dacryoadenitis | Adacm3 | autoimmune dacryoadenitisin | MGI: | 5 | 63.9 cM | Kamo et al. Invest |
| | | MRL mice 1 | 4410451 | | | Ophthalmol Vis Sci 2009; 50: 3257 |

The above-mentioned method may further include inoculating a tumor to the above-mentioned model mouse before the step of administering the antitumor drug and/or the immunotherapy drug. The inoculation of the tumor to the model mouse can produce a model mouse suffering from the tumor.

Examples of the kind of the tumor to be inoculated include, but not limited to, an MRL/N1 cell (PLos ONE 8 (2): e55797.) and an FM3A cell (J Immunol Methods. 2013 Mar. 29; 389 (1-2): 69-78), and the tumor may be appropriately selected by a person skilled in the art. Examples of the site to which the tumor is inoculated include, but not limited to, a lymph node and a flank, and the site may be appropriately selected by a person skilled in the art.

In some embodiments, the present invention provides a method of producing an interstitial pneumonia model mouse, the method including
administering the immune checkpoint inhibitor to a model mouse that is any one of
(i) one or two or more kinds selected from the group consisting of: an MXH7/Mo/Ipr mouse, an MXH10/Mo/Ipr mouse, an MXH28/Mo/Ipr mouse, an MXH36/Mo/Ipr mouse, an MXH41/Mo/Ipr mouse, an MXH43/Mo/Ipr mouse, an MXH51/Mo/Ipr mouse, and an MXH54/Mo/Ipr mouse, or
(ii) one or two or more kinds selected from the group consisting of: an McH/Mo/Ank.Y^{C3H} mouse, an McH/Mo/Ank.Y^{MRL} mouse, and an McH/Mo/Ipr/RA1 mouse.

Examples of the immune checkpoint inhibitor include, but not limited to, inhibitors for PD-1, PD-L1, PD-L2, B7-H3, B7-H4, B7-H5, BTLA, CD80, CD86, CD96, CD47, CD155, CTLA-4, LAG-3, TIGIT, TIM-3, CD111, DNAM-1, Galectin-9, HVEM, Nectin-2, Nectin-3, PVRIG, SIRP alpha, SIRP alpha V2, SIRP gamma, and CD160. The immune checkpoint inhibitor may be an antibody against each of those immune checkpoint proteins. Preferred examples of the immune checkpoint inhibitor include immune checkpoint inhibitors selected from the group consisting of: an anti-CTLA-4 antibody; an anti-PD-1 antibody; and an anti-PD-L1 antibody.

Examples of the route of administration of the immune checkpoint inhibitor to the model mouse include, but not limited to, oral administration, intravenous administration, intralymphatic administration, intraperitoneal administration, transdermal administration, intrathecal administration, intramuscular administration, intranasal administration, transmucosal administration, subcutaneous administration, and transrectal administration. Preferred examples of the route of administration include intravenous administration, intralymphatic administration, and intraperitoneal administration.

The dose of the test substance may be appropriately selected by a person skilled in the art in accordance with the kind of drug. Examples of the dose with respect to the body weight of the model mouse include, but not limited to, from 1 μg/kg to 1,000 mg/kg or from about 1 μg/kg to about 1,000 mg/kg, from 1 μg/kg to 100 μg/kg, from 100 μg/kg to 500 μg/kg, from 500 μg/kg to 1,000 μg/kg, from 1 mg/kg to 10 mg/kg, from 10 mg/kg to 100 mg/kg, from 100 mg/kg to 500 mg/kg, from 200 mg/kg to 300 mg/kg, from 100 mg/kg to 250 mg/kg, from 200 mg/kg to 400 mg/kg, from 250 mg/kg to 500 mg/kg, from 250 mg/kg to 750 mg/kg, from 50 mg/kg to 750 mg/kg, from 1 mg/kg to 10 mg/kg, and from 100 mg/kg to 1,000 mg/kg.

The above-mentioned method may further include inoculating a tumor to the above-mentioned model mouse before the step of administering the immune checkpoint inhibitor.

Examples of the kind of the tumor to be inoculated include, but not limited to, an MRL/N1 cell (PLos ONE 8 (2): e55797.) and an FM3A cell (J Immunol Methods. 2013 Mar. 29; 389 (1-2): 69-78), and the tumor may be appropriately selected by a person skilled in the art. Examples of the site to which the tumor is inoculated include, but not limited to, a lymph node and a flank, and the site may be appropriately selected by a person skilled in the art.

In some model mice each having administered thereto the immune checkpoint inhibitor, interstitial pneumonia may develop as an immune-related adverse effect. To the best of the knowledge of the inventors of the present invention, out of disease model mice produced on the basis of a mouse obtained by the mating of an MRL/Ipr mouse and a C3H/Ipr mouse, no interstitial pneumonia model mouse is present before the filing date of the present application. Accordingly, the above-mentioned method of producing an interstitial pneumonia model mouse is useful in research on such interstitial pneumonia.

In a specific embodiment, in any one of the above-mentioned methods, the model mouse is one or two or more kinds selected from the group consisting of: the MXH7/Mo/Ipr mouse, the MXH10/Mo/Ipr mouse, the MXH28/Mo/Ipr mouse, the MXH36/Mo/Ipr mouse, the MXH41/Mo/Ipr mouse, the MXH43/Mo/Ipr mouse, the MXH51/Mo/Ipr mouse, and the MXH54/Mo/Ipr mouse.

In a specific embodiment, in any one of the above-mentioned methods, the model mouse is one or two or more kinds selected from the group consisting of: the McH/Mo/Ank.Y^{C3H} mouse, the McH/Mo/Ank.Y^{MRL} mouse, and the McH/Mo/Ipr/RA1 mouse.

In some embodiments, the present invention provides a method including administering an immune checkpoint inhibitor to a model mouse that is any one of
(i) one or two or more kinds selected from the group consisting of: an MXH7/Mo/Ipr mouse, an MXH10/Mo/Ipr mouse, an MXH28/Mo/Ipr mouse, an MXH36/Mo/Ipr mouse, an MXH41/Mo/Ipr mouse, an MXH43/Mo/Ipr mouse, an MXH51/Mo/Ipr mouse, and an MXH54/Mo/Ipr mouse, or
(ii) one or two or more kinds selected from the group consisting of: an McH/Mo/Ank.Y^{C3H} mouse, an McH/Mo/Ank.Y^{MRL} mouse, and an McH/Mo/Ipr/RA1 mouse.

Such model mouse is useful in research on interstitial pneumonia.

The disclosures of all the patent applications and literatures cited herein are incorporated herein by reference in their entirety.

The present invention is more specifically described below by way of Examples, but the present invention is not limited thereto.

### EXAMPLES

### Example 1 Administration of Immune Checkpoint Inhibitor to MXH51/Mo/Ipr Recombinant Inbred Mouse

### (Test Method)

### Anti-PD-1 Antibody Reagent

An anti-PD-1 antibody (1141119, Ultra-LEAF™ Purified anti-mouse CD279 (PD-1) Antibody, BioLegend, San Diego, CA, USA) and an anti-CTLA-4 antibody (106213, Ultra-LEAF™ Purified anti-mouse CD152 Antibody, BioLegend) were used. The antibodies were each diluted with physiological saline to a target concentration.

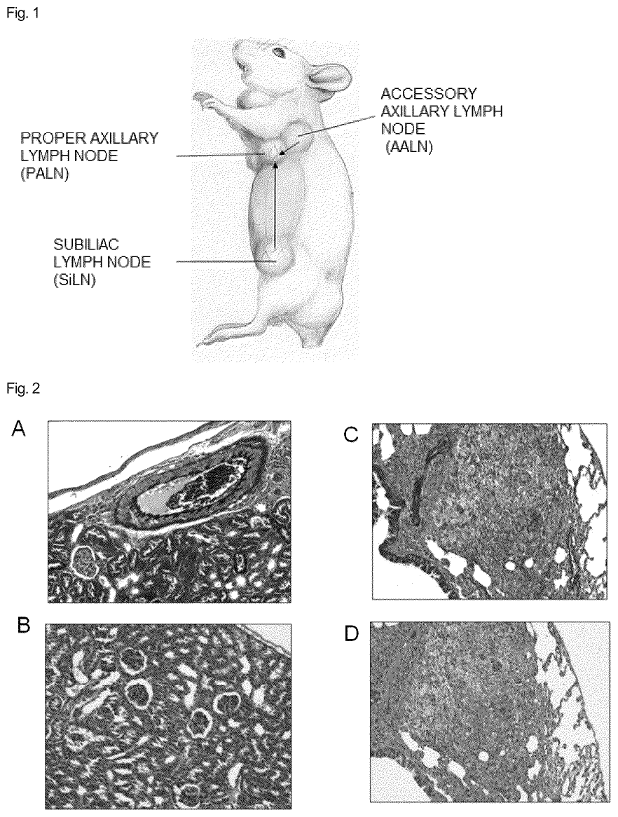

### MXH51/Mo/Ipr Mouse (FIG. 1)

Two male mice aged between 12 weeks and 18 weeks were used for the administration of the anti-PD-1 antibody.

Three male mice and two female mice aged between 12 weeks and 18 weeks were used for the administration of the anti-CTLA-4 antibody.

### Administration to MXH51/Mo/Ipr Mouse

200 Microliters of the anti-PD-1 antibody (1 mg/kg) is manually administered to each of the subiliac lymph nodes (SiLNs) of the mice (male, n=2). The date of administration was defined as day 0T.

Similarly, 200 μL of the anti-CTLA-4 antibody (5 mg/kg) is manually administered to each of the subiliac lymph nodes (SiLNs) of the mice (male: n=3, female: n=2). The date of administration was defined as day 0T.

### Assessment of Volume of Lymph Node

The volumes of a subiliac lymph node (SILN) and a proper axillary lymph node (PALN) were measured with a high-frequency ultrasonic imaging apparatus (VEVO 770, FUJIFILM VisualSonics, Inc., Toronto, ON, Canada). An ultrasonic transducer (704B, center frequency: 40 MHz, FUJIFILM VisualSonics, Inc.) was used in the measurement. The dates of measurement are day 0T, day 4T, day 8T, day 7T, day 11T, day 14T, day 17T, day 24T, day 31T, and day 38T.

### Toxicity Analysis

The experiment was stopped and a mouse was sacrificed in each of the following cases:
a case in which the body weight of the mouse reduced by 10% per week as compared to the initial volume thereof;
a case in which the mouse developed depilation, dyspnea, or diarrhea;
a case in which a rash, suppression by necrosis, a hemodynamic change with ischemia, or the like in a PALN occurred; or
a case in which the daily intake of food or water was recognized, and an abnormality was recognized.

### Histopathological Analysis

A SILN, a PALN, a liver, a lung, a heart, a kidney, a spleen, a pancreas, a large intestine, a salivary gland, a knee joint, and an ankle joint are extracted from each mouse on day 38T.

A paraffin section is produced as a tissue section.
Fixation: 10% formalin (4° C., 4 days, 1 day)
Dehydration: 100% ethanol (normal temperature, 2 days or more) *Each of the knee joint and the ankle joint is immersed in 5% EDTA-Na (shaken at normal temperature for 1 day and then stored at 4° C.) before its paraffin substitution, and is decalcified for up to 3 weeks. Note: the solution was replaced with a new one once or twice a week.

Paraffin replacement and paraffin embedding were performed, and then hematoxylin-eosin (HE) staining (all the organs) or Elastica-Masson (EM) staining (the lung, the kidney, and the heart) was performed.

### Result

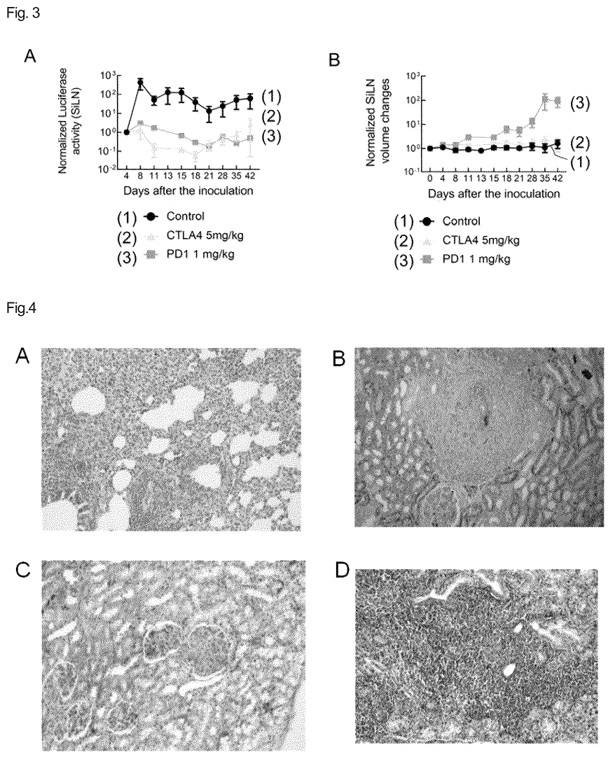

A pathological image when the anti-PD-1 antibody (1 mg/kg) is administered to each of the MXH51/Mo/Ipr mice is shown. The administration of the anti-PD-1 antibody (1 mg/kg) did not develop vasculitis, nephritis, or the like (FIG. 2(A) and FIG. 2(B), but developed interstitial pneumonia (FIG. 2(C) and FIG. 2(D)).

Development frequencies are shown in Table 2. The administration of the anti-PD-1 antibody or the anti-CTLA-4 antibody induced the development of interstitial pneumonia.

TABLE 2
Development frequency of irAE induced by administration of anti-
PD-1 antibody or anti-CTLA-4 antibody to MXH51/Mo/Ipr mouse

| | | Administration of | | | Administration of | | |
|---|---|---|---|---|---|---|---|
| | | anti-PD-1 antibody | | | anti-CTLA-4 antibody | | |
| | | MXH51/Mo/Ipr | | | | | |
| | Immune- | 1 mg/kg | | | 5 mg/kg | | |
| | related | | | Development | | | Development |
| | adverse | | | ratio in | | | ratio in |
| Organ | effect | Male | Female | all mice | Male | Female | all mice |
| Lung | Pneumonia | 2/2 | | 2/2 | 3/3 | 2/2 | 5/5 |

### Example 2 Tumor Transplantation and Administration of Immune Checkpoint Inhibitor to MXH10/Mo/Ipr Recombinant Inbred Mouse

### (Test Method)

### Anti-PD-1 Antibody Reagent

An anti-PD-1 antibody (1141119, Ultra-LEAF™ Purified anti-mouse CD279 (PD-1) Antibody, BioLegend, San Diego, CA, USA) and an anti-CTLA-4 antibody (106213, Ultra-LEAF™ Purified anti-mouse CD152 Antibody, BioLegend) were used. The antibodies were each diluted with physiological saline to a target concentration.

### MXH10/Mo/Ipr Mouse (FIG. 1)

Two female mice aged between 12 weeks and 18 weeks were used for the administration of the anti-PD-1 antibody.

One male mouse aged between 12 weeks and 18 weeks was used for the administration of the anti-CTLA-4 antibody.

### Cells

FM3A-Luc mouse breast cancer cells each strongly expressing a luciferase gene were used. A cell culture solution is formed of RPMI-1640 (Sigma-Aldrich Co. LLC, St Louis, MO, USA). 10% (v/V) fetal bovine serum (Hyclone Laboratories, Inc., Logan, UT, USA), 1% (v/v) L-glutamine-penicillin-streptomycin (Sigma-Aldrich Co. LLC), and 0.5 mg/ml G-418 (Sigma-Aldrich Co. LLC). The cells were cultured at 37° C. in 5% CO₂. The medium was replaced with a new one every 4 to 5 days, and the cells were subcultured three times before being used in an experiment.

### Tumor Transplantation

The tumor cells were transplanted to the right SILN of each of the mice (FIG. 1). To recognize that no mycoplasma infection was present, the presence or absence of the infection was recognized with a mycoplasma detection kit (MycoAlert Mycoplasma Detection Kit, Basel, Switzerland). After the recognition of the fact that no infection was present, the concentration of the cell suspension was adjusted to 1.1×10⁶ cells/mL with phosphate-buffered saline (PBS, Sigma-Aldrich Co. LLC). Further, the suspension was diluted threefold with 400 mg/ml Matrigel (Collaborative Biomedical Products, Inc., Bedford, MA, USA) to have a final concentration of 3.3×10⁵ cells/mL. Next, 60 μL of the cell solution was manually administered to the right SiLN of each of the mice. The date of transplantation is defined as day-4T.

### Administration of Anti-CTLA-4 Antibody or Anti-PD-1 Antibody

On the 4th day (day 0T) after the tumor transplantation, 200 μL of the anti-PD-1 antibody (1 mg/kg) or the anti-CTLA-4 antibody (10 mg/kg) was manually administered to the right subiliac lymph node (SILN).

### Recognition of Tumor Proliferation

An in vivo luminous imaging system (IVIS Lumina LT Series III; PerkinElmer, Inc., Waltham, MA, USA) was used. The dates of measurement are the 0th day (day-4T), the 4th day (day 0T), the 8th day (day 4T), the 11th day (day 7T), the 15th day (day 11T), the 18th day (day 14T), the 21st day (day 17T), the 28th day (day 24T), the 35th day (day 31T), and the 42nd day (day 38T) after the tumor transplantation.

### Assessment of Volume of Lymph Node

The volumes of a subiliac lymph node (SiLN) and a proper axillary lymph node (PALN) were measured with a high-frequency ultrasonic imaging apparatus (VEVO 770, FUJIFILM VisualSonics, Inc., Toronto, ON, Canada). An ultrasonic transducer (center frequency: 40 MHz, 704B, FUJIFILM VisualSonics, Inc.) was used in the measurement. The dates of measurement are the 0th day (day-4T), the 4th day (day 0T), the 8th day (day 4T), the 11th day (day 7T), the 15th day (day 11T), the 18th day (day 14T), the 21st day (day 17T), the 28th day (day 24T), the 35th day (day 31T), and the 42nd day (day 38T) after the tumor transplantation.

### Toxicity Analysis

The experiment was stopped and a mouse was sacrificed in each of the following cases:
a case in which the body weight of the mouse reduced by 10% per week as compared to the initial volume thereof;
a case in which the mouse developed depilation, dyspnea, or diarrhea;
a case in which a rash, suppression by necrosis, a hemodynamic change with ischemia, or the like in a PALN occurred; or
a case in which the daily intake of food or water was recognized, and an abnormality was recognized.

### Histopathological Analysis

A SILN, a PALN, a liver, a lung, a heart, a kidney, a spleen, a pancreas, a large intestine, a salivary gland, a knee joint, and an ankle joint are extracted from each mouse on day 38T.

A paraffin section is produced as a tissue section.

### In Case of Paraffin-embedded Specimen

Fixation: 10% formalin (4° C., 4 days, 1 day)
Dehydration: 100% ethanol (normal temperature, 2 days or more) *Each of the knee 1.5 joint and the ankle joint is immersed in 5% EDTA-Na (shaken at normal temperature for 1 day and then stored at 4° C.) before its paraffin substitution, and is decalcified for up to 3 weeks. Note: the solution was replaced with a new one once or twice a week.
Paraffin replacement: Excelsior is used (1 day).
Paraffin embedding: Histocenter is used.

### In Case of Frozen Sample

Medium for freezing and thawing: OCT
Hematoxylin-eosin (HE) staining (all the organs) or Elastica-Masson (EM) staining (the lung, the kidney, and the heart) was performed as staining.

### Result

The development ratio of an irAE in each organ, which develops at the time of the administration of the anti-PD-1 antibody or the anti-CTLA-4 antibody to each of the cancer-carrying lymph nodes (SiLNs) of the MXH10/Mo/Ipr mice, is shown in Table 3. The administration of the anti-PD-1 antibody developed interstitial pneumonia at 100%, but did not develop any irAE in any other organ. The administration of the anti-CTLA-4 antibody did not develop any irAE in any organ.

TABLE 3
Development frequency of irAE induced by administration of anti-
PD-1 antibody or anti-CTLA-4 antibody to MXH10/Mo/Ipr mouse

| | | Administration of | | | Administration of | | |
|---|---|---|---|---|---|---|---|
| | | anti-PD-1 antibody | | | anti-CTLA-4 antibody | | |
| | | MXH10/Mo/Ipr | | | | | |
| | Immune- | 1 mg/kg | | | 10 mg/kg | | |
| | related | | | Development | | | Development |
| | adverse | | | ratio in | | | ratio in |
| Organ | effect | Male | Female | all mice | Male | Female | all mice |

| | | | | | |
|---|---|---|---|---|---|
| Lung | Pneumonia | 2/2 | 2/2 | 0/1 | 0/1 |

### Example 3 Tumor Transplantation and Administration of Immune Checkpoint Inhibitor to MX10/Mo/Ipr Recombinant Inbred Mouse

### (Test Method)

### Anti-PD-1 Antibody Reagent

An anti-PD-1 antibody (1141119, Ultra-LEAF™ Purified anti-mouse CD279 (PD-1) Antibody, BioLegend, San Diego, CA, USA) and an anti-CTLA-4 antibody (106213, Ultra-LEAF™ Purified anti-mouse CD152 Antibody, BioLegend) were used. The antibodies were each diluted with physiological saline to a target concentration.

### MXH10/Mo/Ipr Mouse

Three male mice and ten female mice aged between 12 weeks and 18 weeks were used for physiological saline (control group). One male mouse and six female mice aged between 12 weeks and 18 weeks were used for the administration of the anti-PD-1 antibody.

Four female mice aged between 12 weeks and 18 weeks were used for the administration of the anti-CTLA-4 antibody.

### Cells

FM3A-Luc mouse breast cancer cells each strongly expressing a luciferase gene were used. A cell culture solution is formed of RPMI-1640 (Sigma-Aldrich Co. LLC, St Louis, MO, USA), 10% (v/v) fetal bovine serum (Hyclone Laboratories, Inc., Logan, UT, USA), 1% (v/V) L-glutamine-penicillin-streptomycin (Sigma-Aldrich Co. LLC), and 0.5 mg/ml G-418 (Sigma-Aldrich Co. LLC). The cells were cultured at 37° C. in 5% CO₂. The medium was replaced with a new one every 4 to 5 days, and the cells were subcultured three times before being used in an experiment.

### Tumor Transplantation

The tumor cells were transplanted to the right SILN of each of the mice (FIG. 1). To recognize that no mycoplasma infection was present, the presence or absence of the infection was recognized with a mycoplasma detection kit (MycoAlert Mycoplasma Detection Kit, Basel, Switzerland). After the recognition of the fact that no infection was present, the concentration of the cell suspension was adjusted to 1.1×10⁶ cells/mL with phosphate-buffered saline (PBS, Sigma-Aldrich Co. LLC). Further, the suspension was diluted threefold with 400 mg/ml Matrigel (Collaborative Biomedical Products, Inc., Bedford, MA, USA) to have a final concentration of 3.3×10⁵ cells/mL. Next, 60 UL of the cell solution was manually administered to the right SILN of each of the mice. The date of transplantation is defined as day-4T.

### Administration of Anti-CTLA-4 Antibody or Anti-PD-1 Antibody

200 μL of the anti-PD-1 antibody (1 mg/kg) or the anti-CTLA-4 antibody (5 mg/kg) was manually administered to the right subiliac lymph node (SiLN). The date of administration is defined as day 0T.

### Recognition of Tumor Proliferation

An in vivo luminous imaging system (IVIS Lumina LT Series III; PerkinElmer, Inc., Waltham, MA, USA) was used. The dates of measurement are the 0th day (day-4T), the 4th day (day 0T), the 8th day (day 4T), the 11th day (day 7T), the 15th day (day 11T), the 18th day (day 14T), the 21st day (day 17T), the 28th day (day 24T), the 35th day (day 31T), and the 42nd day (day 38T) after the tumor transplantation.

### Assessment of Volume of Lymph Node

The volumes of a subiliac lymph node (SILN) and a proper axillary lymph node (PALN) were measured with a high-frequency ultrasonic imaging apparatus (VEVO 770, FUJIFILM VisualSonics, Inc., Toronto, ON, Canada), An ultrasonic transducer (704B, center frequency: 40 MHz, FUJIFILM VisualSonics, Inc.) was used in the measurement. The dates of measurement are the 0th day (day-4T), the 4th day (day 0T), the 8th day (day 4T), the 11th day (day 7T), the 15th day (day 11T), the 18th day (day 14T), the 21st day (day 17T), the 28th day (day 24T), the 35th day (day 31T), and the 42nd day (day 38T) after the tumor transplantation.

### Toxicity Analysis

The experiment was stopped and a mouse was sacrificed in each of the following cases:
a case in which the body weight of the mouse reduced by 10% per week as compared to the initial volume thereof;
a case in which the mouse developed depilation, dyspnea, or diarrhea;
a case in which a rash, suppression by necrosis, a hemodynamic change with ischemia, or the like in a PALN occurred; or
a case in which the daily intake of food or water was recognized, and an abnormality was recognized.

### Histopathological Analysis

A SILN, a PALN, a liver, a lung, a heart, a kidney, a spleen, a pancreas, a large intestine, a salivary gland, a knee joint, and an ankle joint are extracted from each mouse on day 38T.

A paraffin section is produced as a tissue section.

### In Case of Paraffin-Embedded Specimen

Fixation: 10% formalin (4° C., 4 days, 1 day)
Dehydration: 100% ethanol (normal temperature, 2 days or more) *Each of the knee joint and the ankle joint was immersed in 5% EDTA-Na (shaken at normal temperature for 1 day and then stored at 4° C.) before its paraffin substitution, and was decalcified for up to 3 weeks. Note: the solution was replaced with a new one once or twice a week.
Paraffin replacement: Excelsior is used (1 day).
Paraffin embedding: Histocenter is used.

### In Case of Frozen Sample

Medium for freezing and thawing: OCT
Hematoxylin-eosin (HE) staining (all the organs) or Elastica-Masson (EM) staining (the lung, the kidney, and the heart) was performed as staining.

### Result

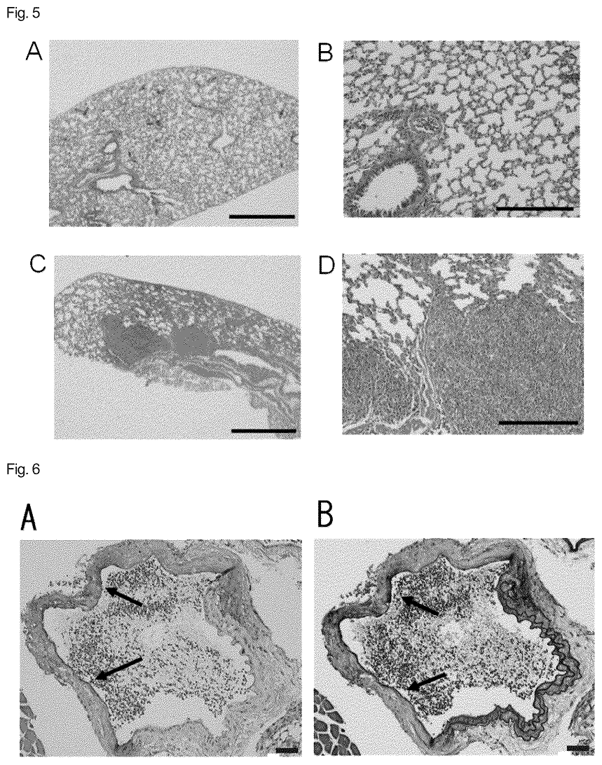

As a result of the administration of each of the anti-CTL4A antibody and the anti-PD-1 antibody to each of MXH10/Mo/Ipr mice, as shown in FIG. 3(A), in the control group (physiological saline alone) to which no immune checkpoint inhibitor had been administered, luciferase activity reflecting an increase in number of tumors increased, but in each of the anti-CTL4A antibody-administered group and the anti-PD-1 antibody-administered group, the luciferase activity was suppressed. The foregoing shows that the tumor-suppressing actions of the anti-CTL4A antibody and the anti-PD-1 antibody are effective. Further, an investigation on a change in volume of the subiliac lymph node (SILN) showed that substantially no change was observed in each of the control group and the anti-CTLA-4 antibody-administered group after the inoculation, but the volume of the SiLN increased in the anti-PD-1 antibody-administered group (FIG. 3(B)).

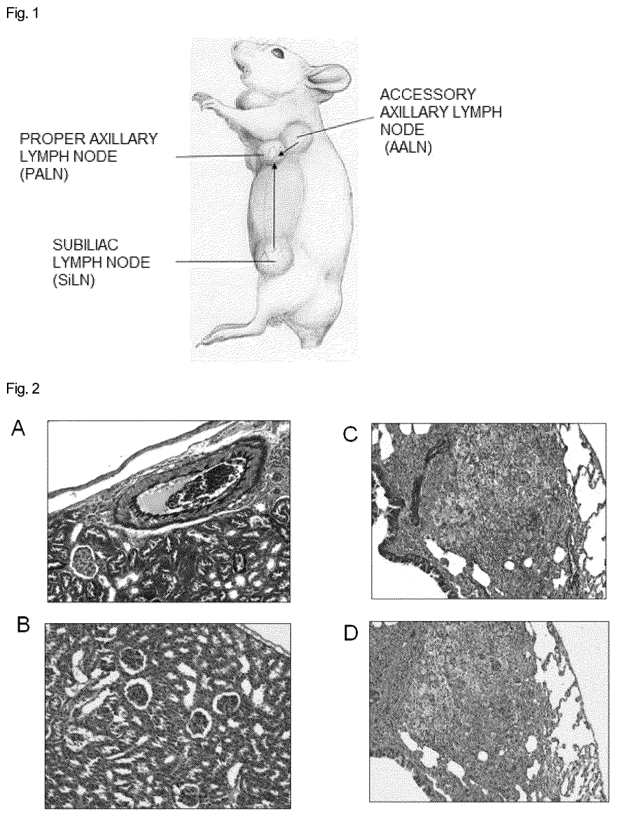

The tissue staining of the anti-CTL4A antibody-administered group and the anti-PD-1 antibody-administered group showed that interstitial pneumonia occurred in the anti-PD-1 antibody-administered group (FIG. 4(A)), and vasculitis, glomerulonephritis, and sialoadenitis developed in the anti-CTL4A antibody-administered group (FIG. 4(B), FIG. 4(C), and FIG. 4(D)).

### Example 4 Occurrence Ratios of Immune Adverse Effect in MXH10/Mo/Ipr Mouse with Respect to Administration of Different Concentrations of Anti-PD-1 Antibody

### (Test Method)

A test was performed by the same method as that of Example 2 unless otherwise described below.

MXH10/Mo/Ipr mice were used.

LM8-luc tumor cells were transplanted as tumor cells to the right SILN of each of the mice. The date of transplantation is defined as day-4T. On day 0T, the anti-PD-1 antibody is administered to the right SILNs of the mice at concentrations of 0.5 mg/kg (male: n=2, female: n=0), 1 mg/kg (male: n=1, female: n=4), 5 mg/kg (male: n=1, female: n=0), and 10 mg/kg (male: n=0, female: n=1). Organs were extracted from the mice on day 38T. Particularly in the case of 1 mg/kg, the development ratio of vasculitis in a kidney increased (Table 4).

TABLE 4
Development of irAEs in MXH10/Mo/Ipr mice by different concentrations
of immune checkpoint inhibitor (anti-PD-1 antibody)

| | Immune- | 0.5 mg/kg | | | 1 mg/kg | | | 5 mg/kg | | |
|---|---|---|---|---|---|---|---|---|---|---|
| | related | | | All | | | All | | | All |
| | adverse | Male | Female | mice | Male | Female | mice | Male | Female | mice |
| Organ | effect | (n = 2) | (n = 0) | (n = 2) | (n = 1) | (n = 4) | (n = 5) | (n = 1) | (n = 0) | (n = 1) |
| Lung | Pneumonia | 0/2 | 0/0 | 0/2 | 0/1 | 1/4 | 1/5 | 0/1 | 0/0 | 0/1 |
| Kidney | Nephritis | 0/2 | 0/0 | 0/2 | 0/1 | 1/4 | 1/5 | 0/1 | 0/0 | 0/1 |
| | Vasculitis | 0/2 | 0/0 | 0/2 | 1/1 | 3/4 | 4/5 | 0/1 | 0/0 | 0/1 |

| | | | | | Aggregation without | | |
|---|---|---|---|---|---|---|---|
| | | | | | consideration of | | |
| | Immune- | 10 mg/kg | | | concentrations | | |
| | related | | | All | | | All |
| | adverse | Male | Female | mice | Male | Female | mice |
| Organ | effect | (n = 0) | (n = 1) | (n = 1) | (n = 4) | (n = 5) | (n = 9) |
| Lung | Pneumonia | 0/0 | 0/1 | 0/1 | 0/4 | 1/5 | 1/9 |
| Kidney | Nephritis | 0/0 | 1/1 | 1/1 | 0/4 | 2/5 | 2/9 |
| | Vasculitis | 0/0 | 1/1 | 1/1 | 1/4 | 4/5 | 5/9 |

### Example 5 Occurrence Ratios of Immune Adverse Effect in MXH10/Mo/Ipr Mouse with Respect to Administration of Different Concentrations of Anti-CTL4-A Antibody

### (Test Method)

A test was performed by the same method as that of Example 2 unless otherwise described below.

MXH10/Mo/Ipr mice were used.

LM8-luc tumor cells were transplanted as tumor cells to the right SILN of each of the mice. The date of transplantation is defined as day-4T. On day 0T, the anti-CTLA-4 antibody is administered to the right SILNs of the mice at concentrations of 1 mg/kg (male: n=2, female: n=1), 5 mg/kg (male: n=1, female: n=4), and 10 mg/kg (male: n=2, female: n=3). Organs were extracted from the mice on day 38T. Particularly in the case of 5 mg/kg, the development ratio of vasculitis in a kidney increased (Table 5).

TABLE 5
Development of irAEs in MXH10/Mo/lpr mice by different concentrations of immune checkpoint inhibitor (anti-CTLA-4 antibody)

| | | | | | | | | | | | Aggregation without | | |
|---|---|---|---|---|---|---|---|---|---|---|---|---|---|
| | | | | | | | | | | | consideration of | | |
| | Immune- | 1 mg/kg | | | 5 mg/kg | | | 10 mg/kg | | | concentrations | | |
| | related | | | All | | | All | | | All | | | All |
| | adverse | Male | Female | mice | Male | Female | mice | Male | Female | mice | Male | Female | mice |
| Organ | effect | (n = 2) | (n = 1) | (n = 3) | (n = 1) | (n = 4) | (n = 5) | (n = 2) | (n = 3) | (n = 5) | (n = 5) | (n = 8) | (n = 13) |
| Lung | Pneumonia | 2/2 | 0/1 | 2/3 | 1/1 | 1/4 | 0.4 | 0/2 | 1/3 | 0.2 | 3/5 | 2/8 | 5/13 |
| Liver | Hepatitis | 1/2 | 0/1 | 1/3 | 0/1 | 0/4 | 0/5 | 0/2 | 0/3 | 0/5 | 1/5 | 0/8 | 1/13 |
| Kidney | Nephritis | 1/2 | 0/1 | 1/3 | 0/1 | 3/4 | 3/5 | 0/2 | 1/3 | 0,2 | 1/5 | 4/8 | 5/13 |
| | Vasculitis | 0/2 | 0/1 | 0/3 | 0/1 | 3/4 | 3/5 | 0/2 | 0/3 | 0/5 | 0/5 | 3/8 | 3/13 |
| Salivary | | 1/2 | 0/1 | 1/3 | 0/0 | 0/0 | 0/5 | 2/2 | 0/3 | 2/5 | 1/5 | 0/4 | 1/9 |
| gland | | | | | | | | | | | | | |
| Spleen | | 0/2 | 0/1 | 0/3 | 0/1 | 0/4 | 0/5 | 0/2 | 0/3 | 0/5 | 0/5 | 0/8 | 0/13 |
| Knee | | 0.5 | 0/1 | 1/3 | 0/0 | 0/3 | 0/3 | 0/1 | 0/1 | 0/2 | 2/5 | 0/5 | 2/10 |
| Ankle | | 0.5 | 0/1 | 1/3 | 0/1 | 0/4 | 0/5 | 0/2 | 0/1 | 0/3 | 1/5 | 0/6 | 1/11 |

### Scoring Criteria

Scoring criteria for the disease states of respective tissues were produced in accordance with Table 6.

TABLE 6
Scoring criteria

| | | |
|---|---|---|
| Lesion | Grade | Evaluation item |

Lymphoid tissue

| | | |
|---|---|---|
| Lesion of lymph node | | |
| Metastasis | 0 | No metastasis/no tumor in site having |
| | | transplanted thereto tumor cell |
| | 1 | Intranodal invasion accounting for ½ or less of |
| | | largest cut surface of lymph node |
| | 2 | Intranodal invasion accounting for more than |
| | | ½ of largest cut surface of lymph node |
| | 3 | Extranodal invasion |
| | 4 | Lymph node is substituted with tumor tissue |
| Necrosis | 0 | No necrosis is observed |
| | 1 | Necrotic focus accounting for ½ or less of |
| | | largest cut surface of lymph node |
| | 2 | Necrotic focus accounting for more than ½ of |
| | | largest cut surface of lymph node |
| | 3 | Necrosis of entirety of lymph node |

Respiratory tissue

| | | |
|---|---|---|
| Lesion of lung | | |
| Metastasis | 0 | No metastasis |
| | 1 | Metastasis is present |
| Interstitial | 0 | No interstitial pneumonia |
| pneumonia | 1 | Localized lesion |
| (excluding | 2 | Multiple lesion |
| alveolar | 3 | Diffuse lesion |
| pneumonia) | | |
| Vasculitis | 0 | Perivascular lymphocytic invasion is |
| | | normal to slight |
| | 1 | Moderate perivascular lymphocytic invasion |
| | | related to breakage of external elastic |
| | | membrane |
| | 2 | Intimal thickening involving breakage of |
| | | external elastic membrane concurring with the |
| | | above-mentioned findings |

Cardiovascular tissue

| | | |
|---|---|---|
| Lesion of heart | | |
| Heart disease | 0 | Normal |
| | 1 | Coronary artery disease/infarction |
| | 2 | Myocarditis/cardiomyopathy |
| | 3 | Endocarditis or valvular heart disease |
| Vasculitis | 0 | Perivascular lymphocytic invasion is normal to |
| | | slight |
| | 1 | Moderate perivascular lymphocytic invasion |
| | | related to breakage of external elastic |
| | | membrane |
| | 2 | Intimal thickening involving breakage of |
| | | external elastic membrane concurring with the |
| | | above-mentioned findings |

Digestive tissue

| | | |
|---|---|---|
| Submaxillitis | | |
| Sialoadenitis | 0 | No inflammatory cell invasion |
| | 1 | Monocyte invasion localized to |
| | | periphery of excretory duct is |
| | | observed, but no breakage is |
| | | observed in parenchyma, excretory |
| | | duct, or acinar cell |
| | 2 | Marked breakage of parenchyma |
| | | involving mononuclear cell invasion |
| | | reaching salivary gland parenchyma |
| | 3 | Significant breakage of salivary |
| | | gland parenchyma involving tubular |
| | | proliferation and granulomatous |
| | | lesion |
| Parotitis | | |
| Sialoadenitis | 0 | No inflammatory cell invasion |
| | 1 | Monocyte invasion localized to |
| | | periphery of excretory duct is |
| | | observed, but no breakage is |
| | | observed in parenchyma, excretory |
| | | duct, or acinar cell |
| | 2 | Marked breakage of parenchyma |
| | | involving mononuclear cell invasion |
| | | reaching salivary gland parenchyma |
| | 3 | Significant breakage of salivary |
| | | gland parenchyma involving tubular |
| | | proliferation and granulomatous |
| | | lesion |
| Lesion of large intestine | | |
| | 0 | No abnormal finding |
| | 1 | Inflammatory lesion |
| | 2 | Inflammatory polyp |
| | 3 | Adenoma |
| | 4 | Malignant tumor |
| Lesion of liver | | |
| Metastasis | 0 | No metastasis |
| | 1 | Metastasis is present |
| Hepatitis | 0 | No hepatitis |
| | 1 | Hepatitis is present |
| Pancreatitis | | |
| Pancreatitis | 0 | No inflammatory cell invasion |
| | 1 | Monocyte invasion localized to |
| | | periphery of excretory duct is |
| | | observed, but no breakage is |
| | | observed in parenchyma, excretory |
| | | duct, or acinar cell |
| | 2 | Marked breakage of parenchyma |
| | | involving mononuclear cell invasion |
| | | reaching pancreatic parenchyma |
| | 3 | Significant breakage of salivary |
| | | gland parenchyma involving tubular |
| | | proliferation and granulomatous |
| | | lesion |

Urinary tissue

| | | |
|---|---|---|
| Lesion of kidney | | |
| Metastasis | 0 | No metastasis |
| | 1 | Metastasis is present |
| Glomerulonephritis | 0 | No glomerulonephritis |
| | 1 | Proliferation of glomerular cell and |
| | | inflammatory cell invasion |
| | 2 | Membranous proliferation, |
| | | lobulation, or deposition of hyaline |
| | | substance |
| | 3 | Crescent formation or spherical |
| | | hyalinization |
| Vasculitis | 0 | Perivascular lymphocytic invasion is |
| | | normal to slight |
| | 1 | Moderate perivascular lymphocytic |
| | | invasion related to breakage of |
| | | external elastic membrane |
| | 2 | Intimal thickening involving |
| | | breakage of external elastic |
| | | membrane concurring with the |
| | | above-mentioned findings |

Skeletal tissue

| | | |
|---|---|---|
| Gonarthritis | | |
| Arthritis | 0 | No arthritis |
| | 1 | Thickening and proliferation of |
| | | synovium |
| | 2a | Granulomatous lesion of |
| | | subsynovial tissue concurring with |
| | | grade 1 |
| | 2b | Fibrous change of synovial tissue |
| | | that does not involve bone change |
| | | concurring with grade 1 |
| | 3a | Pannus formation concurring with |
| | | grade 2 |
| | 3b | Ankylosis |
| Podarthritis | | |
| Arthritis | 0 | No arthritis |
| | 1 | Thickening and proliferation of |
| | | synovium |
| | 2a | Granulomatous lesion of |
| | | subsynovial tissue concurring with |
| | | grade 1 |
| | 2b | Fibrous change of synovial tissue |
| | | that does not involve bone change |
| | | concurring with grade 1 |
| | 3a | Pannus formation concurring with |
| | | grade 2 |
| | 3b | Ankylosis |

### Example 6 Sexual Dimorphism of Occurrence of irAE by Anti-CTLA-4 Antibody

The same test method as that of Example 1 was used.

In the case of the administration of the anti-PD-1 antibody to MXH10/Mo/Ipr mice (male: n=3, female: n=0), a male mouse (n=1) was used, and in the case of the administration of the anti-CTLA-4 antibody, male mice (n=2) were used.

In the case of the administration of the anti-PD-1 antibody to MXH51/Mo/Ipr mice (male: n=5, female: n=2), male mice (n=2) were used, and in the case of the administration of the anti-CTLA-4 antibody, male mice (n=3) and the two female mice were used. The disease states of the respective tissues were subjected to scoring in accordance with the scoring criteria shown in Table 6, and the average of the scores was calculated.

As shown in Table 7, the anti-PD-1 antibody induced an immune-related adverse effect (pneumonia) in each of the MXH10/Mo/Ipr mice and the MXH51/Mo/Ipr mice. The anti-PD-1 antibody tended to induce the immune-related adverse effect (pneumonia) in the MXH51/Mo/Ipr mice to a larger extent.

TABLE 7
Sexual dimorphism of development of irAE by anti-CTLA-4 antibody

| | MXH10/Mo/IPr | | | | MXH51/Mo/IPr | | | |
|---|---|---|---|---|---|---|---|---|
| Drug/ | Male | | Female | | Male | | Female | |
| strain | (n) | Pneumonia | (n) | Pneumonia | (n) | Pneumonia | (n) | Pneumonia |

| | | | | | | | |
|---|---|---|---|---|---|---|---|
| Anti-PD-1 | 1 | 3 | 0 | 1 | 2 | 0 | |
| antibody | | | | 1 | 1 | 0 | |
| Anti- | 2 | 0 | 0 | 2 | 2 | 1 | 1 |
| CTLA-4 | | | | 1 | 1 | 1 | 3 |
| antibody | | | | | | | |

### Example 7 Identification of Gene Related to Adverse Effect in MXH Recombinant Inbred Mouse

According to the results of Example 1, the administration of the anti-PD-1 antibody to each of the MXH51/Mo/Ipr mice does not develop vasculitis or nephritis, but develops interstitial pneumonia. According to the results of Example 2, the administration of the anti-PD-1 antibody to each of the MXH10/Mo/Ipr mice develops all of vasculitis, glomerulonephritis, and interstitial pneumonia. Those results are shown in Table 8.

TABLE 8
Difference in lesion development of irAE induced
by administration of anti-PD-1 antibody between
MXH51/Mo/Ipr mice and MXH10/Mo/Ipr mice

| | MXH10/Mo/IPr | MXH51/Mo/Ipr |
|---|---|---|
| Vasculitis | Yes | No |
| Nephritis | Yes | No |
| Interstitial pneumonia | Yes | Yes |

Yes: Development of irAE is present
No: No development of irAE

The MXH10/Mo/Ipr mice and the MXH51/Mo/Ipr mice are different from each other in presence or absence of the development of vasculitis. Herein, when reference is made to the strain distribution pattern (SDP) sheet of part of the analysis results of genomic information on the fourth chromosomes of McH/Ipr-RA1 mice, the MXH10/Mo/Ipr mice, and the MXH51/Mo/Ipr mice by a microsatellite method shown in Table 9, in the D4Mit89 regions of the fourth chromosomes, a difference in genotype is found between the MXH10/Mo/Ipr mice and the MXH51/Mo/Ipr mice. In addition, it has been known that the regions each correspond to a CD72 gene and the CD72 is related to vasculitis.

Accordingly, it is recognized that the development of vasculitis in each of the MXH10/Mo/Ipr mice and the MXH51/Mo/Ipr mice is related to the CD72 gene of the fourth chromosome.

In addition, the MXH10/Mo/Ipr mice and the MXH51/Mo/Ipr mice are different from each other in presence or absence of the development of nephritis. Herein, when reference is made to the strain distribution pattern (SDP) sheet of part of the analysis results of the fifth chromosomes of McH/Ipr-RA1 mice, the MXH10/Mo/Ipr mice, and the MXH51/Mo/Ipr mice shown in Table 10, in the D5Mit115 regions of the fifth chromosomes, a difference in genotype is found between the MXH10/Mo/Ipr mice and the MXH51/Mo/Ipr mice. In addition, it has been known that the regions each correspond to an osteopontin gene and the osteopontin is related to nephritis.

Accordingly, it is recognized that the development of nephritis in each of the MXH10/Mo/Ipr mice and the MXH51/Mo/Ipr mice is related to the osteopontin gene of the fifth chromosome.

The genomic analysis results of the respective mice are not limited to those obtained by the microsatellite method, and the chromosomes thereof can be easily analyzed by a genomic analysis method well known to a person skilled in the art, such as genome-wide analysis by SNP typing, genomic copy number analysis by an array CGH method, or exome analysis.

TABLE 9
Strain distribution pattern (SDP) sheet of part of analysis results of genomic information on fourth
chromosomes of McH/Ipr-RA1 mice, MXH10/Mo/Ipr mice, and MXH51/Mo/Ipr mice by microsatellite method

| | | McH/Ipr-RA1 | MXH10/Ipr | MXH51/Ipr | Lesion/symbol |
|---|---|---|---|---|---|
| Primer | Gene locus (cM) | MRL:M/C3H:C | MRL:M/C3H:C | MRL:M/C3H:C | |
| D4Mit266 | 6.5 | MM | CC | CC | |
| D4Mit213 | 13.3 | MM | | | Vasculitis/Aaom1 |
| D4Mit89 | 19.8 | MM | MM | CC | Vasculitis/Arvm1 |
| D4Mit271 | 20.8 | | MM | CC | |
| | 22 | | | | Glomerulonephritis/Agnm1 |
| D4Mit197 | 28.6 | MM | MM | CC | |
| D4Mit17 | 31.4 | MM | MM | CC | |
| D4Mit80 | 37.7 | MM | CC | CC | |
| D4Mit15 | 42.6 | MM | CC | CC | |
| D4Mit255 | 48.5 | MM | CC | CC | |
| D4Mit187 | 49 | MM | | | |
| D4Mit306 | 50.8 | | CC | CC | |
| | 51 | | | | Sialoadenitis/Asm2 |
| | 53 | | | | Glomerulonephritis/Agnm2 |
| D4Mit125 | 58 | MM | CC | CC | Vasculitis/Arvm1 |
| D4Mit338 | 59.5 | MM | CC | CC | |
| D4Mit232 | 71 | MM | MM | MM | |
| D4Mit259 | 76 | MM | CC | CC | |
| D4Mit131 | 81.5 | MM | | MM | |

MM: MRL/MRL
Homozygote
CC: C3H/C3H
Homozygote

TABLE 10
Strain distribution pattern (SDP) sheet of part of analysis results of fifth
chromosomes of McH/Ipr-RA1 mice, MXH10/Mo/Ipr mice, and MXH51/Mo/Ipr mice

| | | McH/Ipr-RA1 | MXH10/Ipr | MXH51/Ipr | Lesion/symbol |
|---|---|---|---|---|---|
| Primer | Gene locus (cM) | MRL:M/C3H:C | MRL:M/C3H:C | MRL:M/C3H:C | |
| D5Mit145 | 0 | MM | MM | MM | |
| D5Mit61 | 8 | MM | MM | | |
| D5Mit74 | 11 | MM | MM | MM | |
| D5Mit149 | 20 | MM | MM | | |
| D5Mit200 | 36 | MM | MM | CC | |
| D5Mit259 | 50 | MM | MM | CC | |
| D5Mit115 | 56 | MM | | | Glomerulonephritis/Agnm3 |
| D5Mit239 | 58 | MM | MM | CC | |
| D5Mit136 | 65 | MM | MM | CC | Vasculitis/Aevm2 |
| D5Mit130 | 72 | MM | MM | CC | |
| D5Mit292 | 83 | MM | MM | MM | |
| D5Mit169 | 94 | MM | MM | MM | |

MM: MRL/MRL Homozygote
CC: C3H/C3H Homozygote

### Example 8 Occurrence of Adverse Effect in McH/Mo/Ipr/RA1 Recombinant Congenic Mouse

### (Test Method)

### Anti-PD-1 Antibody Reagent

An anti-PD-1 antibody (1141119, Ultra-LEAF™ Purified anti-mouse CD279 (PD-1) Antibody, BioLegend, San Diego, CA, USA) was used. The antibody was diluted with physiological saline to a target concentration.

### McH/Mo/Ipr/RA1 Mouse

Five female mice aged 22 weeks were used for the anti-PD-1 antibody-administered group and four male mice aged 25 weeks were used for the control group (physiological saline alone).

### Administration of Anti-PD-1 Antibody

The date of administration is defined as day 0T. On day 0T, 200 UL of the anti-PD-1 antibody (1 mg/kg) was manually administered to the right subiliac lymph node (SILN).

### Assessment of Volume of Lymph Node

The volumes of a subiliac lymph node (SILN) and a proper axillary lymph node (PALN) were measured with a high-frequency ultrasonic imaging apparatus (VEVO 770, FUJIFILM VisualSonics, Inc., Toronto, ON, Canada). An ultrasonic transducer (center frequency: 40 MHz, 704B, FUJIFILM VisualSonics, Inc.) was used in the measurement. The dates of measurement are day 0T, day 4T, day 8T, day 11T, day 15T, day 18T, day 21T, day 28T, day 35T, day 38T, and day 42T.

### Toxicity Analysis

The experiment was stopped and a mouse was sacrificed in each of the following cases:
a case in which the body weight of the mouse reduced by 10% per week as compared to the initial volume thereof;
a case in which the mouse developed depilation, dyspnea, or diarrhea;
a case in which a rash, suppression by necrosis, a hemodynamic change with ischemia, or the like in a PALN occurred; or
a case in which the daily intake of food or water was recognized, and an abnormality was recognized.

### Histopathological Analysis

A SILN, a PALN, a liver, a lung, a heart, a kidney, a spleen, a pancreas, a large intestine, a salivary gland, a knee joint, and an ankle joint are extracted from each mouse on day 38T.

A paraffin section is produced as a tissue section.

### In Case of Paraffin-Embedded Specimen

Fixation: 10% formalin (4° C., 4 days, 1 day)
Dehydration: 100% ethanol (normal temperature, 2 days or more) *Each of the knee joint and the ankle joint was immersed in 5% EDTA-Na (shaken at normal temperature for 1 day and then stored at 4° C.) before its paraffin substitution, and was decalcified for up to 3 weeks. Note: the solution was replaced with a new one once or twice a week.
Paraffin replacement: Excelsior is used (1 day).
Paraffin embedding: Histocenter is used.

### In Case of Frozen Sample

Medium for freezing and thawing: OCT
Hematoxylin-eosin (HE) staining (all the organs) or Elastica-Masson (EM) staining (the lung, the kidney, and the heart) was performed as staining.

### Result

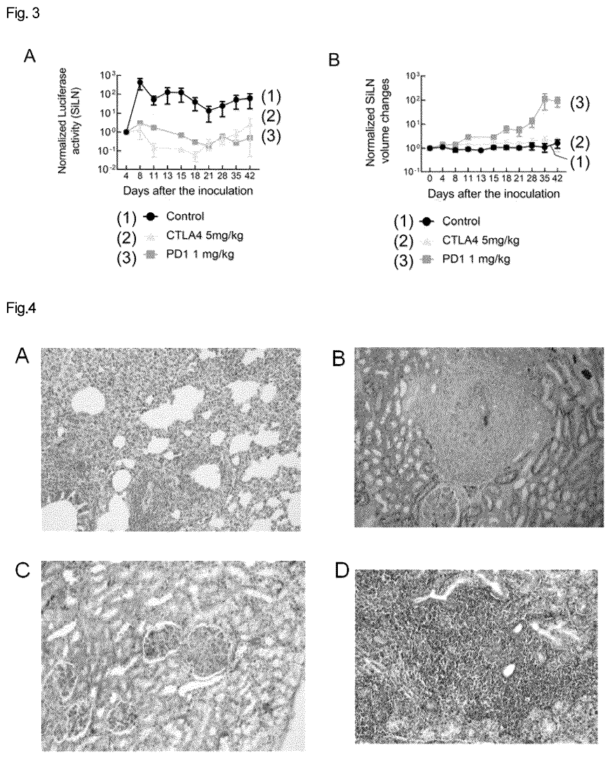

The administration of the anti-PD-1 antibody to each of the subiliac lymph nodes of the McH/Ipr-RA1 mice developed interstitial pneumonia that was an irAE (FIG. 5(C) and FIG. 5(D)). According to the pathological scoring criteria (Table 6) concerning pneumonia used in the laboratory of the applicants, the score of the control group (n=4) is zero, but the score of the immune checkpoint inhibitor (ICI)-administered group (n=5) is 1.4, and hence there is a statistically significant difference (P<0.0046) therebetween. The result was conceived to originate from the manifestation of an inflammatory lesion in a disease-sensitive gene that the McH/Ipr-RA1 mice each potentially had by the anti-PD-1 antibody serving as an ICI.

### Example 9 Administration of Immune Checkpoint Inhibitor to MXH41/Mo/Ipr Recombinant Inbred Mouse

### (Test Method)

### Anti-PD-1 Antibody Reagent

An anti-PD-1 antibody (1141119, Ultra-LEAFIM Purified anti-mouse CD279 (PD-1) Antibody, BioLegend, San Diego, CA, USA) was used. The antibody was diluted with physiological saline to a target concentration.

### Administration to MXH41/Mo/Ipr Mouse

Administration was manually performed to each of the subiliac lymph nodes (SILNs) of the MXH41/Mo/Ipr mice (female, n=3; male, n=2). The date of administration was defined as day 0T.

### Assessment of Volume of Lymph Node

The volumes of a subiliac lymph node (SILN) and a proper axillary lymph node (PALN) were measured with a high-frequency ultrasonic imaging apparatus (VEVO 770, FUJIFILM VisualSonics, Inc., Toronto, ON, Canada). An ultrasonic transducer (704B, center frequency: 40 MHz, FUJIFILM VisualSonics, Inc.) was used in the measurement. The dates of measurement are day 0T, day 4T, day 8T, day 11T, day 15T, day 18T, day 21T, day 28T, day 35T, day 38T, and day 42T.

### Toxicity Analysis

The experiment was stopped and a mouse was sacrificed in each of the following cases:
a case in which the body weight of the mouse reduced by 10% per week as compared to the initial volume thereof;
a case in which the mouse developed depilation, dyspnea, or diarrhea;
a case in which a rash, suppression by necrosis, a hemodynamic change with ischemia, or the like in a PALN occurred; or
a case in which the daily intake of food or water was recognized, and an abnormality was recognized.

### Histopathological Analysis

A SILN, a PALN, a liver, a lung, a heart, a kidney, a spleen, a pancreas, a large intestine, a salivary gland, a knee joint, and an ankle joint are extracted from each mouse on day 38T.

A paraffin section is produced as a tissue section.
Fixation: 10% formalin (4° C., 4 days, 1 day)
Dehydration: 100% ethanol (normal temperature, 2 days or more) *Each of the knee joint and the ankle joint is immersed in 5% EDTA-Na (shaken at normal temperature for 1 day and then stored at 4° C.) before its paraffin substitution, and is decalcified for up to 3 weeks. Note: the solution was replaced with a new one once or twice a week.

Paraffin replacement and paraffin embedding were performed, and then hematoxylin-eosin (HE) staining (all the organs) or Elastica-Masson (EM) staining (the lung, the kidney, and the heart) was performed.

### Result

When the anti-PD-1 antibody (1 mg/kg) was administered to each of the MXH41/Mo/Ipr mice, necrosis or a defect that was conceived to result from vasculitis was observed in each of the forelegs of two mice out of the three female mice, and rubor or swelling was observed in one mouse out thereof. In addition, foreleg defects were observed in two mice out of the two male mice.

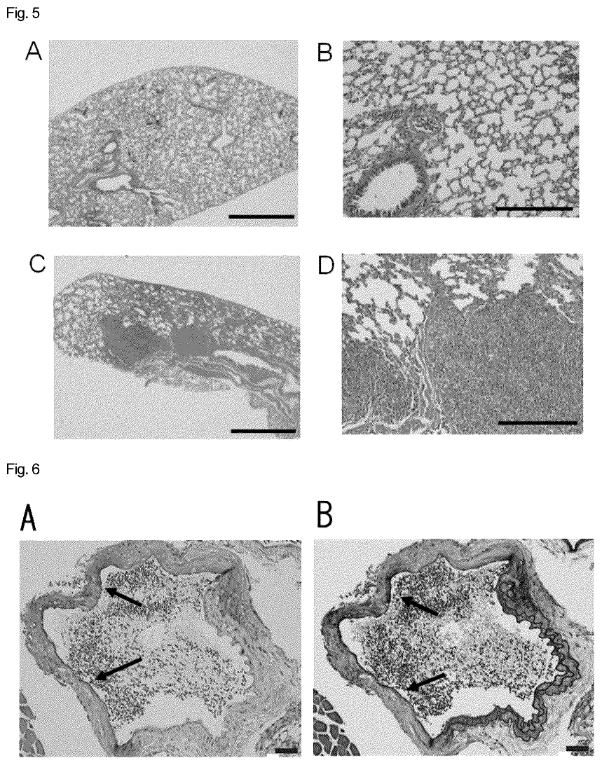

A pathological image of the necrotizing vasculitis of a mouse obtained as follows is shown in each of FIG. 6: the anti-PD-1 antibody is administered to the subiliac lymph node of an MXH41/Mo-Ipr/Ipr mouse to cause necrosis in an upper limb thereof. All the layers of the endodermis, internal elastic membrane, media, and adventitia of an arterial wall present in the upper limb causing the necrosis fall into necrosis (arrows). While FIG. 6(A) shows HE staining, FIG. 6(B) shows EM staining, and a bar in the figure corresponds to 50 μm.

1. A method of assessing an adverse effect induced by an antitumor drug and/or an immune checkpoint inhibitor, the method comprising:
administering the antitumor drug and/or the immune checkpoint inhibitor to a model mouse that is any one of
(i) one or two or more kinds selected from the group consisting of: an MXH7/Mo/Ipr mouse, an MXH10/Mo/Ipr mouse, an MXH28/Mo/Ipr mouse, an MXH36/Mo/Ipr mouse, an MXH41/Mo/Ipr mouse, an MXH43/Mo/Ipr mouse, an MXH51/Mo/Ipr mouse, and an MXH54/Mo/Ipr mouse, or
(ii) one or two or more kinds selected from the group consisting of: an McH/Mo/Ank.Y^{C3H} mouse, an McH/Mo/Ank.Y^{MRL} mouse, and an McH/Mo/Ipr/RA1 mouse; and
assessing the adverse effect induced by the antitumor drug and/or the immune checkpoint inhibitor.

2. The method according to claim 1, wherein the antitumor drug and/or the immune checkpoint inhibitor is the immune checkpoint inhibitor.

3. The method according to claim 1, wherein the adverse effect is interstitial pneumonia, sialoadenitis, vasculitis, arthritis, glomerulonephritis, dacryoadenitis, or a combination of two or more thereof.

4. A method of screening an antitumor drug and/or an immune checkpoint inhibitor, the method comprising:
administering a test substance that is the antitumor drug and/or the immune checkpoint inhibitor to a model mouse that is any one of
(i) one or two or more kinds selected from the group consisting of: an MXH7/Mo/Ipr mouse, an MXH10/Mo/Ipr mouse, an MXH28/Mo/Ipr mouse, an MXH36/Mo/Ipr mouse, an MXH41/Mo/Ipr mouse, an MXH43/Mo/Ipr mouse, an MXH51/Mo/Ipr mouse, and an MXH54/Mo/Ipr mouse, or
(ii) one or two or more kinds selected from the group consisting of: an McH/Mo/Ank.Y^{C3H} mouse, an McH/Mo/Ank.Y^{MRL} mouse, and an McH/Mo/Ipr/RA1 mouse; and
assessing the presence or absence of an adverse effect in the model mouse after the administration of the test substance.

5. The method according to claim 2, wherein the antitumor drug and/or the immune checkpoint inhibitor is the immune checkpoint inhibitor.

6. The method according to claim 2, wherein the adverse effect is interstitial pneumonia, sialoadenitis, vasculitis, arthritis, glomerulonephritis, dacryoadenitis, or a combination of two or more thereof.

7. A method of identifying a chromosome region related to an adverse effect induced by administration of an antitumor drug and/or an immunotherapy drug, the method comprising:
administering the antitumor drug and/or the immunotherapy drug to each of a plurality of kinds of model mice each of which is any one of
(i) one or two or more kinds selected from the group consisting of: an MXH7/Mo/Ipr mouse, an MXH10/Mo/Ipr mouse, an MXH28/Mo/Ipr mouse, an MXH36/Mo/Ipr mouse, an MXH41/Mo/Ipr mouse, an MXH43/Mo/Ipr mouse, an MXH51/Mo/Ipr mouse, and an MXH54/Mo/Ipr mouse, or
(ii) one or two or more kinds selected from the group consisting of: an McH/Mo/Ank.Y^{C3H} mouse, an McH/Mo/Ank.Y^{MRL} mouse, and an McH/Mo/Ipr/RA1 mouse; and
identifying a chromosome region related to a difference in adverse effect induced by the administration of the antitumor drug and/or the immunotherapy drug between the plurality of kinds of model mice.

8. The method according to claim 7, wherein the adverse effect is interstitial pneumonia, sialoadenitis, vasculitis, arthritis, glomerulonephritis, dacryoadenitis, or a combination of two or more thereof.

9. A method of identifying a chromosome region related to an immune adverse effect induced by administration of an immune checkpoint inhibitor, the method comprising:
administering the immune checkpoint inhibitor to a model mouse that is any one of
(i) one or two or more kinds selected from the group consisting of: an MXH7/Mo/Ipr mouse, an MXH10/Mo/Ipr mouse, an MXH28/Mo/Ipr mouse, an MXH36/Mo/Ipr mouse, an MXH41/Mo/Ipr mouse, an MXH43/Mo/Ipr mouse, an MXH51/Mo/Ipr mouse, and an MXH54/Mo/Ipr mouse, or
(ii) one or two or more kinds selected from the group consisting of: an McH/Mo/Ank.Y^{C3H} mouse, an McH/Mo/Ank.Y^{MRL} mouse, and an McH/Mo/Ipr/RA1 mouse; and
identifying the chromosome region related to the immune adverse effect induced by the administration of the immune checkpoint inhibitor.

10. The method according to claim 9, wherein the adverse effect is interstitial pneumonia, sialoadenitis, vasculitis, arthritis, glomerulonephritis, dacryoadenitis, or a combination of two or more thereof.

11. A method of producing an interstitial pneumonia model mouse, the method comprising:
administering an immune checkpoint inhibitor to a model mouse that is any one of
(i) one or two or more kinds selected from the group consisting of: an MXH7/Mo/Ipr mouse, an MXH10/Mo/Ipr mouse, an MXH28/Mo/Ipr mouse, an MXH36/Mo/Ipr mouse, an MXH41/Mo/Ipr mouse, an MXH43/Mo/Ipr mouse, an MXH51/Mo/Ipr mouse, and an MXH54/Mo/Ipr mouse, or
(ii) one or two or more kinds selected from the group consisting of: an McH/Mo/Ank.Y^{C3H} mouse, an McH/Mo/Ank.Y^{MRL} mouse, and an McH/Mo/Ipr/RA1 mouse.

12. The method according to claim 1, further comprising inoculating a tumor to the model mouse before the administration step.

13. The method according to claim 1, wherein the model mouse is one or two or more kinds selected from the group consisting of: the MXH7/Mo/Ipr mouse, the MXH10/Mo/Ipr mouse, the MXH28/Mo/Ipr mouse, the MXH36/Mo/Ipr mouse, the MXH41/Mo/Ipr mouse, the MXH43/Mo/Ipr mouse, the MXH51/Mo/Ipr mouse, and the MXH54/Mo/Ipr mouse.

14. The method according to claim 1, wherein the model mouse is one or two or more kinds selected from the group consisting of: the McH/Mo/Ank.Y^{C3H} mouse, the McH/Mo/Ank.Y^{MRL} mouse, and the McH/Mo/Ipr/RA1 mouse.

15. A model mouse suffering from interstitial pneumonia, the model mouse being any one of
(i) one or two or more kinds selected from the group consisting of: an MXH7/Mo/Ipr mouse, an MXH10/Mo/Ipr mouse, an MXH28/Mo/Ipr mouse, an MXH36/Mo/Ipr mouse, an MXH41/Mo/Ipr mouse, an MXH43/Mo/Ipr mouse, an MXH51/Mo/Ipr mouse, and an MXH54/Mo/Ipr mouse, or
(ii) one or two or more kinds selected from the group consisting of: an McH/Mo/Ank.Y^{C3H} mouse, an McH/Mo/Ank.Y^{MRL} mouse, and an McH/Mo/Ipr/RA1 mouse.
